SEQUENCE LISTING (20 sequences)

SEQ ID NO: 1 - DNA, 555 bp, Artificial
  FIII9-10 Pro(1408) protein; Fibronectin mutant cDNA encoding the region spanning the FIII9-10 domain and contains the mutation Leu(1408) to Pro(1408)
ggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccccattgattggccaacaatcaacagtttctgatgttccgagggacctggaagttgttgctgcgacccccaccagcctactgatcagctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacataa

SEQ ID NO: 2 - protein, 184 aa, Artificial
  FIII9-10 Pro(1408); Fibronectin mutant polypeptide of the region spanning the FIII9-10 domain with the mutation Leu(1408) to Pro(1408)
GLDSPTGIDFSDITANSFTVHWIAPRATITGYRIRHHPEHFSGRPREDRVPHSRNSITLTNLTPGTEYVVSIVALNGREESPPLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 3 - DNA, 555 bp, Artificial
  FIII9-10 Ile(1358) protein; Fibronectin mutant cDNA encoding the region spanning the FIII9-10 domain and contains the mutation Arg(1358) to Ile(1358)
ggtcttgattccccaactggcattgacttttctgatattactgccaactcttttactgtgcactggattgctcctcgagccaccatcactggctacatcatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacataa

SEQ ID NO: 4 - protein, 184 aa, Artificial
  FIII9-10 Ile(1358); Fibronectin mutant polypeptide of the region spanning the FIII9-10 domain with the mutation Arg (1358) to Ile(1358)
GLDSPTGIDFSDITANSFTVHWIAPRATITGYIIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 5 - DNA, 555 bp, Artificial
  FIII9-10 Pro(1408) Ile(1358) protein; Fibronectin mutant cDNA encoding the region spanning the FIII9-10 domain and contains the double mutation Leu(1408) to Pro(1408) and Arg(1358) to Ile(1358)
ggtcttgattccccaactggcattgacttttctgatattactgccaactcttttactgtgcactggattgctcctcgagccaccatcactggctacatcatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccccattgattggccaacaatcaacagtttctgatgttccgagggacctggaagttgttgctgcgacccccaccagcctactgatcagctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacataa

SEQ ID NO: 6 - protein, 184 aa, Artificial
  FIII9-10 Pro(1408) Ile(1358) protein; Fibronectin mutant polypeptide of the region spanning the FIII9-10 domain with the double mutation at Leu(1408) to Pro(1408) and Arg(1358) to Ile(1358)
GLDSPTGIDFSDITANSFTVHWIAPRATITGYIIRHHPEHFSGRPREDRVPHSRNSITLTNLTPGTEYVVSIVALNGREESPPLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 7 - protein, 90 aa, Mus musculus
GLDSPTGFDSSDITANSFTVHWVAPRAPITGYIIRHHAEHSVGRPRQDRVPPSRNSITLTNLNPGTEYVVSIIAVNGREESPPLIGQQAT

SEQ ID NO: 8 - protein, 90 aa, Homo sapiens
GLDSPTGIDFSDITANSFTVHWIAPRATITGYRIRHHPEHFSGRPREDRVPHSRNSITLTNLTPGTEYVVSIVALNGREESPLLIGQQST

SEQ ID NO: 9 - DNA, 21 bp, Artificial
  Forward oligonucleotide primer
gggtcttgattccccaactgg

SEQ ID NO: 10 - DNA, 25 bp, Artificial
  Reverse oligonucleotide primer
ttattactccagagtggtgacaaca

SEQ ID NO: 11 - DNA, 21 bp, Artificial
  Forward oligonucleotide primer
gccattgtctccaccaacaaa

SEQ ID NO: 12 - DNA, 26 bp, Artificial
  Reverse oligonucleotide primer
ttattatgttcggtaattaatggaaa

SEQ ID NO: 13 - DNA, 38 bp, Artificial
  Oligonucleotide primer
cgatgcggtaccgctagcggtcttgattccccaactgg

SEQ ID NO: 14 - DNA, 35 bp, Artificial
  Oligonucleotide primer
cgcaagcttttatgttcggtaattaatggaaattg

SEQ ID NO: 15 - DNA, 34 bp, Artificial
  Oligonucleotide primer
ggcagagaggaaagtcccccattgattggccaac

SEQ ID NO: 16 - DNA, 34 bp, Artificial
  Oligonucleotide primer
gttggccaatcaatgggggactttcctctctgcc

SEQ ID NO: 17 - DNA, 34 bp, Artificial
  Oligonucleotide primer
ccaccatcactggctacatcatccgccatcatcc

SEQ ID NO: 18 - DNA, 34 bp, Artificial
  Oligonucleotide primer
ggatgatggcggatgatgtagccagtgatggtgg

SEQ ID NO: 19 - DNA, 33 bp, Artificial
  Oligonucleotide primer
ccaactggcattgactcttctgatattactgcc

SEQ ID NO: 20 - DNA, 33 bp, Artificial
  Oligonucleotide primer
ggcagtaatatcagaagagtcaatgccagttgg

(19) United States
(12) Patent Application Publication
(10) Pub. No.: US 2005/0118661 A1
(43) Pub. Date: Jun. 2, 2005

(54) Fibronectin variants and screening methods

(76) Inventors: Helen Mardon, Oxford (GB); David Barlow, Glasgow (GB); Ian Sargent, Oxford (GB); Christopher Van derWalle, Blantyre (GB)

Correspondence Address: FOLEY & LARDNER LLP, 111 HUNTINGTON AVENUE, 26TH FLOOR, BOSTON, MA, 02199-7610, US

(21) Appl. No.: 10/895,590

(22) Filed: Jul. 21, 2004

Related U.S. Application Data
(63) Continuation of application No. PCT/GB03/00212, filed on Jan. 21, 2003.

(30) Foreign Application Priority Data
Jan. 21, 2002 (GB) GB0201273.0

(51) Int. Cl. G01N 33/567; G01N 33/53; G01N 33/537; G01N 33/543

(52) U.S. Cl. 435/7.92

(57) ABSTRACT

A method is provided for assessing fertility status in female patient which comprises determining the level of fibronectin isoform EDIIIA+ and/or EDIIIB+ in an isolated endometrial sample within the implantation window time for the patient and correlating the appearance of said fibronectin isoform with fertility status. Fibronectin variants useful as contraceptive agents are also described.

The present invention is concerned with fibronectin proteins and their uses. In particular, the invention is concerned with assays based on interactions with fibronectin proteins. In addition, novel variants of fibronectins are also provided.

Fibronectins are large, dimeric multidomain, glycoproteins (˜220 kDa) that are abundant in the extra cellular matrix (ECM) of many cell types. Each FN subunit comprises a series of FI, FII and FIII domains that, alone or in combination with adjacent domains, form separable functional units with binding activity for cells and other components of the ECM. At least 20 possible isoforms of human fibronectin can be expressed as a result of alternative splicing of the primary RNA transcript. Two FIII domains, EDIIIA and EDIIIB that reside each side of the central cell binding domain (CCBD), can be included or excluded in the mature protein as a result of exon skipping. Alternative splicing of EDIIIA and EDIIIB is tightly regulated according to cell type and developmental stage.

Implantation of the human blastocyst is likely to involve the regulated cellular processes of adhesion, invasion and proliferation of the embryonic trophectoderm as it attaches to, and penetrates the endometrial lumenal epithelium and underlying stroma. The regulation of these diverse processes depends upon coordinated signals from both soluble factors and extracellular matrix (ECM) molecules. Studies in rodents would suggest that there is considerable cross signalling from the implanting embryo to the endometrium and vice versa.

The ECM is a dynamic and complex mixture of a number of molecules including large proteins and glycoproteins such as fibronectins (FN), laminins, collagens, vitronectin and tenascins, and proteoglycans. In addition, some growth factors are sequestered in the ECM by virtue of their ability to bind other ECM molecules such as heparin. However the precise composition of the ECM is dependent upon the cell type and tissue status.

The adhesion of cells to specific components of the ECM, including fibronectin, depends upon interaction of the ligands with the integrin family of cell surface receptors (Hynes et al, Fibronectins, 1990). Integrins are heterodimers of one α and one β subunit. More than sixteen α and eight β subunits have been identified so far, some of which can heterodimerize with multiple partners, whilst others form functional heterodimers with just one partner. Fibronectin can interact with a number of different integrins, including α5β1, which only binds fibronectin, and αν heterodimers such as ανβ3 and ανβ5, that also interact with other ECM ligands. In vitro studies have shown that the binding of fibronectin to integrins triggers signalling events inside the cell that result in modulation of cellular activities, for example changes in cell shape, gene expression and organisation of the actin cytoskeleton. In addition, ligand binding elicits signals from inside the cell that result in clustering of integrins on the cell surface and formation of focal adhesion complexes.

Studies of the localization of adhesion molecules in the endometrium and blastocyst suggest functions for cell-cell and cell-matrix interactions in the progression of the human embryo through the implantation process. Specific ECM molecules, such as laminin and collagen type IV, have been shown to be modulated in the endometrium throughout the menstrual cycle (Aplin et al, Cell. Tiss. Res., 253, 231-240, 1998; Bulleti et al., Cancer 62, 142-149, 1988; Faber et al, Am. J. Pathol, 124, 384-391, 1986; Vollmer et al, Lab. Invest., 62, 725-730, 1990; Sasauo et al, Mod. Pathol., 6, 323-326, 1993). It has been shown that the EDIIIB isoform is highly expressed during angiogenesis both in neoplastic and in normal tissues, and in the functional layer of endometrium during the proliferative phase (Zardi et al., Int. J. Cancer, 59, 612-618, 1994).

A number of ECM receptors exhibit strict cell-type specific expression in the epithelial or stromal compartments of the endometrium. Integrin subunit β5 is expressed on the apical surface of the lumenal endometrium and has thus been implicated in implantation (Alpin et al., Mol. Human. Reprod. 2, 527-534, 1996) and integrin subunits αν, β3, α1 and α4 exhibit a sharp increase in expression in the endometrium at the time of implantation (Lessey et al, J. Clin. Invest., 90, 188-195, 1997; Rai et al, J. Pathol., 180, 181-187, 1996). Furthermore, assessment of integrin expression in the epithelium of endometrium obtained from women with unexplained infertility suggests that the integrin subunit β3 has reduced expression (Lessey et al, J. Clin. Endrocrionl. Metab., 79, 643-649, 1994) and that α4 is reduced in the lumenal epithelium, during the window of implantation, compared to fertile controls (Klentzeris et al, Hum. Reprod., 8, 1223-1230, 1993).

Expression of integrin subunits α3, αν, β1, β3 and β5 in pre-implantation human blastocysts has been described (Campbell et al, Mol. Hum. Reprod., 10, 1571-1578, 1995). All of these integrin subunits are present at each stage of development and observed from oocyte to pre-hatched blastocyst. However expression of these molecules in the human peri-implantation, hatched blastocysts has not been described. Moreover, the role of fibronectin isoforms in the endometrium during embryo implantation is not fully understood.

The molecular and cellular mechanisms that underpin the process of implantation of the human blastocyst are not known, in spite of the fact that one of the major causes of infertility is failure of the embryo to implant successfully into the endometrium.

Embryo implantation involves three main stages of events. In the first stage the pre-implantation blastocyst and the endometrium are primed for implantation. This involves successful hatching of the blastocyst and expression of molecules on the trophectoderm that are permissive for uterine-embryo interaction. At the same time the endometrium must be receptive for embryo attachment, which, in the human, occurs during a window of implantation from about day 19-22 of the endometrial cycle. Coordination of these events in the two tissues is thus critical. Development to the blastocyst stage appears to be to some extent autonomously driven as blastocyst formation from eggs can be obtained in defined complex serum free medium. There may be considerable cross signalling by soluble factors that facilitate the priming of the blastocyst and endometrium immediately prior to implantation; refs (Ghosh et al, Mol. Hum. Reprod., 4, 733-735, 1998; Paria et al, Proc. Natl. Acad. Sci. 98, 1047-1052, 2001).

During the second stage of implantation the hatched blastocyst attaches to the luminal epithelium of the endometrium, penetrates the epithelium and basement membrane and invades into the underlying stroma. Very little work has been done in this key area in the human mainly because it is not possible to study implantation sites in situ.

The third stage involves extensive trophoblast proliferation and differentiation, and invasion of the stroma resulting in the colonization of the maternal blood supply and establishment of a functional placenta. Most is known about this stage of the implantation process in the human due to the availability of material from first trimester pregnancies. However the molecular and cellular events involved in first trimester placentation are clearly distinct from those involved during the earlier, histiotrophic stages of implantation.

The ECM, integrins and the matrix metalloproteinases (MMPs) and their inhibitors (TIMPS), are likely to have key functions in implantation. The regulated expression of these molecules on the trophectoderm of the hatched blastocyst has been demonstrated in rodents. Tightly coordinated expression of these molecules, together with growth factors, may thus facilitate embryo attachment during the first stage of implantation, and the penetration of the embryo through the epithelial basement membrane of the endometrium and the underlying stromal ECM. Specific ECM molecules may also be induced by growth factors secreted from the endometrial epithelium, as shown for tenascin.

Integrins are developmentally regulated in the mouse embryo. Integrins α5β1, α6β1 and ανβ3 are expressed continuously from fertilisation whereas α2, α6A and α7 (which all bind laminin) are only expressed at the blastocyst stage. The integrin subunit α3 is expressed from the eight cell stage onward but α1 is undetectable until outgrowth of trophoblasts has begun. Also in the mouse, ανβ3 integrin, but not the α5 subunit are localised on the surface of late blastocyst stage embryos (Sutherland et al, Development, 119, 1175-1186, 1993). Less is known about integrin expression on human embryos. Integrins α3β1, ανβ3 and ανβ5 and possibly α6β4 have been detected up to the blastocyst stage (Campbell et al, Mol. Hum. Reprod., 10, 1571-1578, 1995), but there are no reports of integrin expression at the time of blastocyst hatching and attachment.

Fibronectin has been shown previously to be abundant in the extracellular matrix of the human endometrium throughout the menstrual cycle (Aplin et al, Cell. Tiss. Res., 253, 231-240, 1988).

Previous studies in the mouse suggest that FN can support blastocyst attachment and trophoblasts outgrowth (Schultz et al, J. Biol. Chem. 1995, 270, 11522-11531, 1995; Yelian et al, Mol. Reprod. Dev., 41, 435-448, 1995).

The inventors have now identified the function of specific isoforms of FN in implantation of human blastocyst.

The elucidation of the cellular and molecular mechanisms involved in implantation of the human embryo represents a clinically important, if intractable, biological problem. The difficulties encountered in the study of implantation in the human include significant technical limitations that are associated with experimentation of relevant biological material, which must be overcome. The inventors have dissected the function of FN variants in implantation of the human blastocyst and defined a function for endometrial EDIIIA+ and EDIIIB+ FN in secondary implantation events.

The inventors have now found that expression of EDIIIA+ and EDIIIB+ FN is increased in the human endometrium during the window of implantation. A function for specific FN isoforms in implantation is further suggested by the capacity of recombinant EDIIIA+/B+ FN protein fragments to support blastocyst attachment via α5β1 integrin in functional in vitro studies of embryo-fibronectin interactions. The inventors' observations suggest that EDIIIA+ FN and integrin-mediated signalling have a key function in implantation of the human blastocyst.

Based on the above observations, the present inventors have identified assays for screening compounds capable of modulating embryo implantation. Diagnostic assays are also provided.

The present inventors have also identified FIII fibronectin variant fragments; these variants are specially designed for conducting the above tests of the present invention.

The primary cell binding site on fibronectin is the RGD motif on the tenth type III module (FIII10) (Pierschbacher et al., Nature, 309, 30-33, 1984). Cell spreading in response to FIII10 is very poor, however, but progressively increases on addition of modules N-terminal to FIII10 (Obara et al., Cell., 53, 649-657, 1988). This is due to a ‘synergy site’ (PHSRN) in FIII9 and together with FIII10 this module pair forms the ‘central cell binding domain’ which gives near-maximal cell adhesion and spreading activity (Mardon et al., FEBS lett., 340, 197-201, 1994) or inhibition of fibronectin activity (Aota et al., J. Biol. Chem., 269,24756-24761, 1994). The synergy site has recently been more extensively characterised showing that Arg¹³⁷⁴ is key to the synergistic activity (Redick et al., J. Cell Biol., 149, 521-527, 2000). The type III modules of fibronectin all share a common framework (Huber et al., Neuron, 12, 717-731, 1994; Potts et al., Cell Biol., 6, 648-655, 1994) which previously has been determined to high resolution for FIII10 (Main et al., Cell, 71, 671-678, 1992; Dickinson et al., J. Mol. Biol., 236, 1079-1092, 1994). The conformation of the primary binding site, the ‘RGD loop’, in these structures was largely disordered. Furthermore, small, highly mobile, RGD-containing peptides elicited a cell attachment response and were inhibitors of fibronectin adhesive activity (Pierschbacher et al., Nature, 309, 30-33, 1984). However, since a full cell attachment and spreading response was only observed with the intact FIII9-10 pair the spatial relationship between the RGD loop and the ‘synergy binding site’ on FIII9 would appear to be critical. This has indeed been proven (Grant et al., J. biol. Chem., 272, 6159-6166, 1997) and is consistent with the hypothesis that two binding sites exist on the fibronectin cell surface receptor: integrin (Humphries et al., Biochem. Soc. Trans., 28, 311-339, 2000). The crystal structure of FIII7-10 (Leahy et al., Cell, 84, 155-164, 1996) shows the four modules in an extended rod-like structure with the RGD and synergy sites 34 Å apart and on loops protruding some distance from the main body. In the crystal structure the FIII9 and 10 modules appear to interact in a manner which maintains the binding loops in the same plane and maximally exposed, since their calculated tilt and rotation were low when compared to the FIII7-8 and FIII8-9 pairs. A certain degree of mobility is thought to exist between FIII9 and 10, because the calculated FIII9-10 buried surface area was low (333 Å²).

Previous attempts to determine the solution structure of hFIII9-10 have been limited by the unfavourable solution properties of hFIII9-10 which is a consequence of the low thermodynamic stability of FIII9 (Spitzfaden et al., J. Mol. Biol., 265, 565-579, 1997). Therefore, only the assignment of human FIII10 has so far been reported (Main et al., Cell, 71, 611-678, 1992). In equilibrium denaturation experiments, the isolated FIII9 module is much less stable than its FIII10 homologue despite having a backbone rmsd with FIII10 of 0.72 Å in the secondary structure elements of the X-ray structure (Plaxco et al., Proc. Natl. Acad. Sci. USA., 93, 10703-10706, 1997). The large difference of stability between the two modules is reflected in the two-step unfolding curve for FIII9-10. Using NMR data acquired for the FIII9-10 pair in different concentrations of denaturant, Spitzfaden et al., 0.1997, have shown that this is indeed a consequence of the initial unfolding of FIII9 followed by the unfolding of FIII10.

The inventors have now identified series of FIII9-10 variants, based on the differences between the mouse-human FIII9 primary sequences, with the aim of improving the stability of the human FIII9 module but maintaining the functional activity by minimal amino acid substitution. The series of hybrid mouse-human FIII9-10 pairs have greater stability than hFIII9-10. In particular, the conformational stability of the human FIII9 module can be increased two-three fold by substitution of Pro¹⁴⁰⁸ for Leu¹⁴⁰⁸, without any loss of cell attachment. The resulting novel FIII9′-10 variant has good solution properties and can be used as a template on which further mutations can be incorporated to probe the structure-function relationship of the cell binding module of fibronectin.

The inventors have also discovered that FIII9′-10 has improved cell adhesion capacity. This discovery can find a possible application in the inhibition of embryo implantation; also this has wide implications in anti-angiogenic or anti-tumorigenic applications.

Finally, the inventors also propose the use of FIII9-10 variants, in particular FIII9′-10, as delivery agents to integrin-expressing cells.

Accordingly, in a first aspect, the invention provides a method for identifying compounds which are capable of modulating the production of FIII fibronectin isoform, in particular EDIIIA+ or EDIIIB+ fibronectin production, which method comprises:
contacting a FIII fibronectin isoform producing cell with said test compound;
determining the amount of FIII isoform in the presence and absence of the test compound determining the effect of the test compound on the amount of the FIII fibronectin isoform and thereby identifying a compound which modulates the production of the protein.

In particular the method may be used to identify compounds which modulate blastocyst implantation into the endometrium and hence may be a fertility enhancing or contraceptive agent.

In a preferred embodiment the assay is carried out on a yeast cell or a cell of mammalian origin expressing the desired fibronectin isoform or a biologically active form thereof. More preferably, the cell is an isolated human female endometrial stromal cell.

Preferably the fibronectin isoform is detected using an antibody specific for one or more epitopes of EDIIIA+ or EDIIIB+ fibronectin isoform, which antibody is conjugated to a molecule which facilitates its identification when complexed with its target antigen. The molecule may be a revealing label such as a radio isotype, luminescent of fluorescent molecule or an enzyme. Alternatively, the molecule may facilitate attachment on the revealing label.

The method of the invention can be advantageously based upon the western blotting assay or enzymes-linked immunosorbent assay (ELISA), both well-known in the art.

Protocols for performing Western blotting and ELISA assays are well known in the art, such as described in Sambrook at al. (Molecular Cloning: a Laboratory Manual, 1989); representative examples are given in the experimental part included herein.

Western blotting is a well known technique for the analysis and identification or proteins. Generally, the complexes are separated by polyacrylamide gel electrophoresis and then transferred to a neutral cellulose membrane or chemically treated paper to which the proteins bind; preferably, the complexes are transferred to a PVDF (Polyvinylidene Difluoride) membrane. The proteins bound to the membrane are detected by overlaying the appropriate antibody.

ELISAs provide sensitive and quantitative detection of specific antigens or antibodies. A variety of ELISA formats can be employed. Commercially available ELISAs are based on the antibody-sandwich format or double-layer variation. The sandwich ELISA generally requires two antibodies that are directed against a particular antigen. One antibody is passively adsorbed (coated) onto the surface of the wells of an ELISA plate. The wells are then “blocked” with a nonspecific protein solution to keep background levels low. The samples containing the antigen in solution are then added to the wells and incubated for a sufficient amount of time for the antigen to bind to the antibody immobilized on the plate. After washing the wells to remove the unbound reagents, the second antibody is added to the well. This second antibody binds to the immobilized antigen completing the sandwich. The second antibody is detected with an enzyme conjugate specific for the second antibody. Alternatively, the second antibody itself can be labelled for subsequent detection. When the enzyme substrate is added to the wells in the final step, the conjugated enzyme, and therefore the antigen, is detected by observing the calorimetric, flourescent or chemiluminescent reaction products in an appropriate ELISA plate reader. In the double-layer technique, antigen is bound to the plastic surface (test tubes, wells or beads) followed by the test sample containing antibody, then the enzyme conjugate. Incubation complexes with a suitable substrate results in a coloured product which may be measured spectrophotometrically.

Preferably, wells are coated with cells producing the desired fibronectin isoform or a biologically active portion. A sample containing the compound under test is then added to the wells and the plates are incubated to allow time for specific modulation of the fibronectin isoform production. The wells are blocked, for instance with a solution of BSA in PBS. A primary antibody is then added; a suitable labelled antibody for example is IST-9. Many other tag molecules which are equally suitable for this purpose are known in the art and commercially available. The wells are then washed and a secondary antibody with the appropriate specificity and enzyme tag are added to the wells. The enzyme substrate is then added and bound proteins are detected, leading to the determination of the amount of the desired fibronectin isoform production.

The above-described method can be used to screen for compounds that inhibit or enhance the production of fibronectin isoforms EDIIIA+ and/or EDIIIB+, and hence, respectively, for inhibiting or enhancing fertility. The screened compounds can respectively be useful as contraceptive agents or as fertility agents.

It will be appreciated that a wide variety of candidate compounds may be tested in the screening methods of the invention. Suitable test compounds may include, for example, compounds having a known pharmacological or biochemical activity, compounds having no such identified activity and completely new molecules or libraries of molecules such as might be generated by combinatorial chemistry. Compounds which are nucleic acids; including naturally occuring nucleic acids and synthetic analogues, polypeptides or proteins are not excluded.

Typically, compound screening assays involve running a plurality of assay mixtures in parallel with different concentrations of the compound under test. Typically, one of these concentrations serves as a negative control, i.e. zero concentration of test compound.

Compounds which are identifiable as having potential pharmacological activity using the methods of the invention may be used as lead compounds in the further development of drugs with pharmaceutical potential or may themselves be formulated into pharmaceutical compositions.

The above-described methods can be used for screening compounds which can be useful as a contraceptive agent or for treatment of infertility in a female.

According to a further aspect, the invention relates to the compounds identifiable by the above described methods and to contraceptive or pharmaceutical compositions containing them.

In particular, the invention provides a method of producing a composition suitable for treating infertility in a mammalian female or suitable for use as a contraceptive agent in a mammalian female which comprises:
a) carrying out a compound screening method as described above; and
b) formulating any compound capable of modulating production of fibronectin isoform EDIIIA+ and/or EDIIIB+ in a pharmaceutical composition with a pharmaceutically acceptable carrier or diluent.

According to a further aspect, the invention relates to a method of inducing contraception in a mammalian female, preferably a human female, which comprises administering to said female an inhibitor of production of fibronectin isoform EDIIIA+ and/or EDIIIB+ in endometrial cells. Contraceptive compositions comprising said inhibitors formulated with a pharmaceutically acceptable carrier or diluent are also provided.

In yet a further aspect, the invention provides a method of enhancing fertility in a mammalian, preferably human female, which comprises administering to said female an enhancer of production of EDIIIA+ and/or EDIIIB+ in endometriual cells. Pharmaceutical compositions are also provided comprising said enhancer together with a pharmaceutically acceptable carrier or diluent. The use of said enhancer in the manufacture of a medicament for treatment of human female infertility is also provided.

The contraceptive and pharmaceutical compositions of the present invention are advantageously formulated for local application to the endometrium and suitable excipients for this purpose will be well-known to those skilled in the art.

The female reproductive tract is more accessible for specific drug delivery than many other organ systems. The advantages of local application of therapeutic agents to the endometrium are that unwanted effects on other systems that might be induced by systemic drug delivery can be avoided, and the dose can be lower than with systemic administration of drugs. In infertility the need is to deliver drugs directly into the uterus or into the vagina from which natural diffusion may occur. Direct delivery to the uterine cavity is most readily achieved, either by canulation to incorporate a molecule in a liquid medium as already occurs with embryo transfer in IVF with the use of transfer medium, or by instillation through the cervical canal by syringe placed at the cervix without canulation of the uterine cavity. Alternatively drug delivery to the upper vagina and cervix in the form of a viscous gel or tablet is a currently recognised route of drug delivery to this area.

According to a further aspect, the invention provides novel FIII9-10 variants; preferably, said variants include the substitution of Pro¹⁴⁰⁸ for Leu¹⁴⁰⁸ (called herein “FIII9′-10”).

According to a further embodiment, the invention provides the use of FIII9-10 variants, preferably FIII9′-10, as a medicament. These are particularly useful for inhibiting adhesion of cell types to fibronectin.

According to a further embodiment, the invention provides the use of FIII9-10 variants, preferably FIII9′-10, for the preparation of a medicament for use in an indication selected from the group comprising of contraceptive agents, anti-angiogenic agents and anti-tumorigenic agents.

The FIII9′-10 variant may be administered to a mammalian female, preferably human, to induce a contraceptive effect or to mammals preferably human, of either sex to induce an anti-angiogenic or anti-tumourgenic effect.

The invention also includes a contraceptive composition comprising a FIII9-10 variant, preferably. FIII9′-10 and a pharmaceutically acceptable carrier or diluent. Also comprised in the invention are anti-angiogenic and anti-tumourgenic compositions comprising FIII9-10 variants, preferably FIII9′-10 together with a pharmaceutically acceptable carrier or diluent. Again, for contraceptive use, it may be preferable to formulate the composition for delivery via the vagina, for example, as an ointment, cream, gel or liquid or facilitate direct endometrial delivery.

According to a still further embodiment, the invention provides the use of FIII19-10 variants, preferably FIII9′-10, as delivery agents.

Preferably, the above variants can deliver biologically active ingredients or chemical or biological toxins or imaging agents, including peptides and/or chemical compounds to integrin-expressing cells. Integrin-expressing cells include in particular, tumour or endometrium cells. Pharmaceutical compositions comprising an FIII9-10 variant, especially FIII9′-10, as a delivery agent are also within the scope of the present invention.

Suitable active ingredients include those capable of interacting with integrin, preferably α5β1 integrin, in biological/physical systems. More particularly, suitable biologically active ingredients include cell specific toxins (such as anti-cancer agents), growth factor or chemicals having anti-functional ability such as anti-tumorigenic, anti-angiogenic or contraceptive activity.

According to a further aspect, the invention also provides a diagnostic test to assess fertility status in a mammalian, preferably human female. Said test comprises:
determining the level of fibronectin isoform, EDIIIA+ and/or EDIIIB+, in a isolated endometrial sample within implantation window time for the female, and
correlating the appearance of said fibronectin isoform with fertility status.

The above test aims to assess levels of the said fibronectin isoforms, during the implantation window in the endometrium of women and to provide an indication of fertility status. This can be achieved using EDIIIA+ and/or EDIIIB+ isoforms specifically while use of fibronectin itself as a marker is unsuitable because it is abundant throughout the cycle and does not increase at the time of implantation.

As used herein, the term “implantation window” encompasses the window of time-during which the uterine endometrium is receptive to the conceptus; in the human, this occurs in the secretory stage of the menstrual cycle. Implantation is defined as days 6-8 post the day of the luteinising hormone(LH) surge. The implantation window can be estimated on the basis of a regular menstrual pattern as approximately seven days before the expected first day of the menstrual period; alternatively, it may be determined by using the mid-cycle LH urine test (Clear-Plan Styx) to identify the LH surge, starting at day 12 until the surge is detected. Any woman with infertility should have had ovulation confirmed. Starting from an isolated sample, the present method generally involves detection of the desired fibronectin isoform.

Again the determination of the EDIIIA+ and/or EDIIIB+ fibronectin isoform levels may be carried out by any suitable method known to the art including an immunoassay using an antibody as described herein specific for one or more epitopes of EDIIIA+ and/or EDIIIB+. Advantageously detection may be based upon ELISA methodology and/or immunohistochemistry as disclosed above and in the examples and using the appropriate antibodies.

The fibronectin isoform levels can be correlated with endometrial receptivity and likelihood of conception.

The invention also provides kits for detection of EDIIIA+ and/or EDIIIB+ expression in any endometrial sample from a mammalian female, preferably a human female which comprises an antibody specific for one or more epitope of EDIIIA+ and/or EDIIIB+, said antibody being conjugated to a molecule which facilitates its detection when complexed to EDIIIA+ or EDIIIB+. Preferably the antibody is conjugated to a revealing label such as a luminescent or flourescent molecule, radio-isotype or enzyme. Where the revealing label is an enzyme the kit further includes a substrate for the enzyme. As an alternative the antibody may be conjugated to a molecule; for example biotin, which facilitates attachment of a revealing label. The revealing label for attachment may be an enzyme in which case said kit further comprises said enzyme.

The inventors have also constructed fibronectin mutants derived from human fibronectin that have greater stability and biological activity compared to wild type FIII9-10.

These fibronectin cDNAs encode the region spanning the FIII9 to FIII10 domains and contain mutations of in specific amino acids.

According to a further aspect, the invention provides a protein which comprises the amino acid sequence set forth in SEQ ID NO: 2, SEQ ID NO: 4 or SEQ ID NO: 6 or an amino acid sequence which differs from that shown in SEQ ID NO: 2, SEQ ID NO: 4 or SEQ ID NO:6 only in conservative amino acid changes.

Also provided by the invention are nucleic acid sequences which encode the proteins of the invention.

Also provided by the invention are a nucleic acid comprising the sequences of nucleotides set forth in SEQ ID NO:1, SEQ ID NO: 3 or SEQ ID NO: 5.

The splice variants of human FIII fibronectin isoforms were cloned by PCR technology.

The first fibronectin mutant cDNA encodes the region spanning the FIII9 to FIII10 domains and contains the mutation Leu¹⁴⁰⁸ to Pro¹⁴⁰⁸. The nucleotide sequence is set forth in SEQ ID NO:1 and the corresponding amino acid sequence of the recombinant protein (designated FIII9-10 Pro¹⁴⁰⁸) is set forth in SEQ ID NO:2.

The second fibronectin mutant cDNA encodes the region spanning the FIII9 to FIII10 domains and contains mutation Arg¹³⁵⁸ to Ile¹³⁵⁸. The nucleotide sequence is set forth in SEQ ID NO:3 and the corresponding amino acid sequence of the recombinant protein (designated FIII9-10 Ile¹³⁵⁸) is set forth in SEQ ID NO:4.

The third fibronectin mutant cDNA encodes the region spanning the FIII9 to FIII10 domains and contains the double mutation Leu¹⁴⁰⁸ to Pro¹⁴⁰⁸ and Arg¹³⁵⁸ to Ile¹³⁵⁸. The nucleotide sequence is set forth in SEQ ID NO:5 and the corresponding amino acid sequence of the recombinant protein (designated FIII9-10 Pro¹⁴⁰⁸, Ile¹³⁵⁸) is set forth in SEQ ID NO:6.

The nucleic acid molecules according to the invention may, advantageously, be included in a suitable expression vector to express the proteins encoded therefrom in a suitable host. Incorporation of cloned DNA into a suitable expression vector for subsequent transformation of said cell and subsequent selection of the transformed cells is well known to those skilled in the art as provided in Sambrook et al. (1989), molecular cloning, a laboratory manual, Cold Spring Harbour Laboratory Press.

An expression vector according to the invention includes a vector having a nucleic acid according to the invention operably linked to regulatory sequences, such as promoter regions, that are capable of effecting expression of said-DNA fragments. The term “operably linked” refers to a juxtaposition wherein the components described are in a relationship permitting them to function in their intended manner. Such vectors may be transformed into a suitable host cell to provide for expression of a protein according to the invention. Thus, in a further aspect, the invention provides a process for preparing proteins according to the invention which comprises cultivating a host cell, transformed or transfected with an expression vector as described above under conditions to provide for expression by the vector of a coding sequence encoding the protein, and recovering the expressed protein.

The vectors may be, for example, plasmid, virus or phage vectors provided with an origin of replication, and optionally a promoter for the expression of said nucleotide sequence and optionally a regulator of the promoter. The vectors may contain one or more selectable markers, such as, for example, an antibiotic resistance.

Regulatory elements required for expression include promoter sequences to bind RNA polymerase and to direct an appropriate level of transcription initiation and also translation initiation sequences for ribosome binding. For example, a bacterial expression vector may include a promoter such as the lac promoter and for translation initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or be assembled from the sequences described by methods well known in the art.

Nucleic acid molecules according to the invention may be inserted into the vectors described in an antisense orientation in order to provide for the production of antisense RNA. Antisense RNA or other antisense nucleic acids, including antisense peptide nucleic acid (PNA), may be produced by synthetic means.

In accordance with the present invention, a defined nucleic acid includes not only the identical nucleic acid but also any minor base variations including in particular, substitutions in cases which result in a synonymous codon (a different codon specifying the same amino acid residue) due to the degenerate code in conservative amino acid substitutions. The term “nucleic acid sequence” also includes the complementary sequence to any single stranded sequence-given regarding base variations.

The nucleic acid sequences according to the invention may be produced using recombinant or synthetic techniques, such as for example using PCR which generally involves making a pair of primers, which may be from approximately 10 to 50 nucleotides to a region of the gene which is desired to be cloned, bringing the primers into contact with cDNA, or genomic DNA from a human cell, performing a polymerase chain reaction under conditions which brings about amplification of the desired region, isolating the amplified region or fragment and recovering the amplified DNA. Generally, such techniques are well known in the art, such as described in Sambrook et al. (Molecular Cloning: a Laboratory Manual, 1989).

The nucleic acids according to the invention may carry a revealing label. Suitable labels include radioisotopes such as ³²P or ³⁵S, enzyme labels or other protein labels such as biotin or fluorescent markers. Such labels may be added to the nucleic acids or oligonucleotides of the invention and may be detected using known techniques per se.

The protein according to the invention includes all possible amino acid variants encoded by the nucleic acid molecule according to the invention including a protein encoded by said molecule and having conservative amino acid changes. Proteins or polypeptides according to the invention further include variants of such sequences, including naturally occurring allelic variants which are substantially homologous to said proteins or polypeptides. In this context, substantial homology is regarded as a sequence which has at least 70%, preferably 80 or 90% and preferably 95% amino acid homology with the proteins or polypeptides encoded by the nucleic acid molecules according to the invention. The protein according to the invention may be recombinant, synthetic or naturally occurring, but is preferably recombinant.

A further aspect of the invention provides a host cell or organism, transformed or transfected with an expression vector according to the invention. The host cell or organism may advantageously be used in a method of producing protein, which comprises recovering any expressed protein from the host or organism transformed or transfected with the expression vector.

According to a further aspect of the invention there is also provided a transgenic cell, tissue or organism comprising a transgene capable of expressing a protein according to the invention. The term “transgene capable of expressing” as used herein encompasses any suitable nucleic acid sequence which leads to expression of proteins having the same function and/or activity. The transgene, may include, for example, genomic nucleic acid isolated from human cells or synthetic nucleic acid, including DNA integrated into the genome or in an extrachromosomal state. Preferably, the transgene comprises the nucleic acid sequence encoding the proteins according to the invention as described herein, or a functional fragment of said nucleic acid. A functional fragment of said nucleic acid should be taken to mean a fragment of the gene comprising said nucleic acid coding for the proteins according to the invention or a functional equivalent, derivative or a non-functional derivative such as a dominant negative variant, or bioprecusor of said proteins.

The protein expressed by said transgenic cell, tissue or organism or a functional equivalent or bioprecusor of said protein also forms part of the present invention. Recombinant proteins may be recovered and purified from host cell cultures by methods known in the art, including ammonium sulfate or ethanol precipitation, acid extraction, anion or cation exchange chromatography, phosphocellulose, chromatography, hydrophobic interaction chromatography, affinity chromatography, hydroxyapatite chromatography and lectin chromatography.

The protein of the present invention may be a naturally purified product, or a product of chemical synthetic procedures, or produced by recombinant techniques from a prokaryotic or eukaryotic host (for example, by bacterial yeast, higher plant, insect and mammalian cells in culture). Depending upon the host employed in a recombinant production procedure, the expressed protein may lack the initiating methionine residue as a result of post-translational cleavage. Proteins which have been modified in this way are also included within the scope of the invention.

In a still further aspect the invention provides an antibody capable of specifically binding to a protein according to the invention. Preferably the antibody is capable of specifically binding to a protein comprising the sequence of amino acids set forth in SEQ ID NO: 2, SEQ ID NO: 4 or SEQ ID NO: 6. An antibody according to the invention may be raised according to standard techniques well known to those skilled in the art by using the protein of the invention or a fragment or single epitope thereof as the challenging antigen.

A further aspect of the invention comprises a nucleic acid capable of hybridising to the nucleic acids according to the invention, and preferably capable of hybridising to the sequence of nucleotides set forth in SEQ ID NO: 1, SEQ ID NO: 3 or SEQ ID NO: 5, under high stringency conditions. Conditions of stringency are well known to those skilled in the art.

Stringency of hybridisation as used herein refers to conditions under which polynucleic acids are stable. The stability of hybrids is reflected in the melting temperature (Tm) of the hybrids. Tm can be approximated by the formula:
81.5° C.+16.6(log₁₀[Na⁺]+0.41(% G&C)−600/1
wherein 1 is the length of the hybrids in nucleotides. Tm decreases approximately by 1-1.5° C. with every 1% decrease in sequence homology.

The nucleic acid capable of hybridising to nucleic acid molecules according to the invention will generally be at least 70%, preferably at least 80 or 90% and more preferably at least 95% homologous to the nucleotide sequences according to the invention.

The present invention also provides oligonucleotides consisting essentially of at least 10 consecutive nucleotides of a nucleic acid according to the invention and preferably from 10 to 50 consecutive nucleotides of a nucleic acid according to the invention, in particular oligonucleotides fragments from the sequence of nucleotides shown in SEQ ID NO: 1, SEQ ID NO: 3 or SEQ ID NO: 5. These oligonucleotides may, be used as probes or primers to initiate replication, or the like. Oligonucleotides having a defined sequence may be produced according to techniques well known in the art, such as by recombinant or synthetic means. They may also be used in diagnostic kits or the like for detecting the presence of a nucleic acid according to the invention. These tests generally comprise contacting the probe with the sample under hybridising conditions and detecting the presence of any duplex or triplex formation between the probe and any nucleic acid in the sample.

For the avoidance of doubt it is pointed out that all documents cited herein are incorporated herein by reference.

The invention will be further understood with reference to the following experimental examples, together with the accompanying Figures in which:

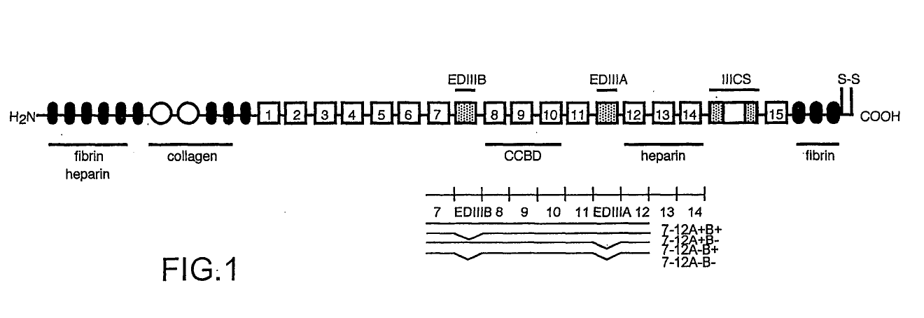

FIG. 1 is a schematic representation of the FN molecule showing FI, FII and FIII structural domains, and the location of EDIIIA and EDIIIB. The binding specificities of FN domains are indicated above. The recombinant variant proteins are shown below.

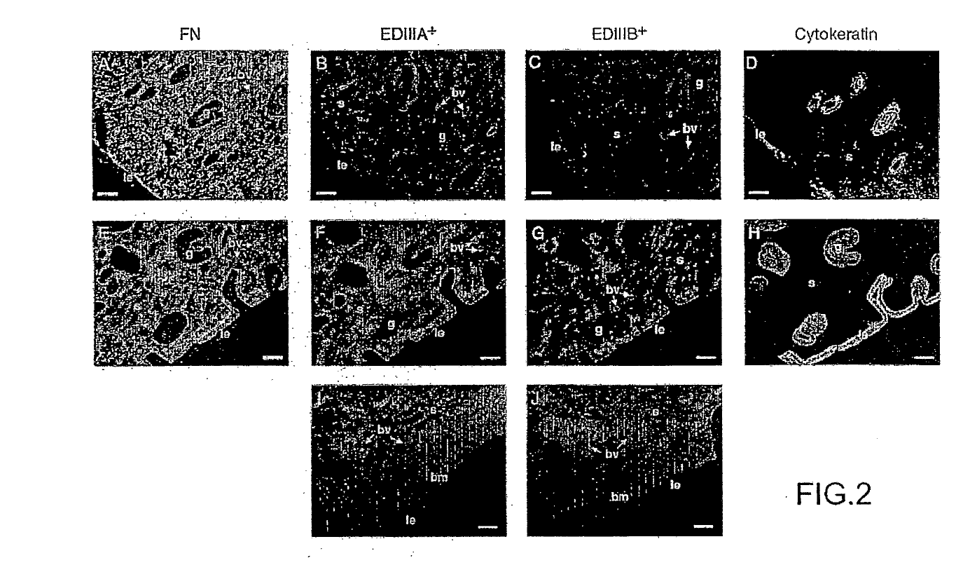

FIG. 2 shows the immunohistochemistry of sections of proliferative stage (A-D) and mid-secretory (Day 22) stage (E-H) human endometrium stained for total FN (FN); EDIIIA+ FN (B<F and I); EDIIIB+ FN(C, G and J), and cytokeratin (D and H). High magnification of F and G are shown in I and J, respectively.

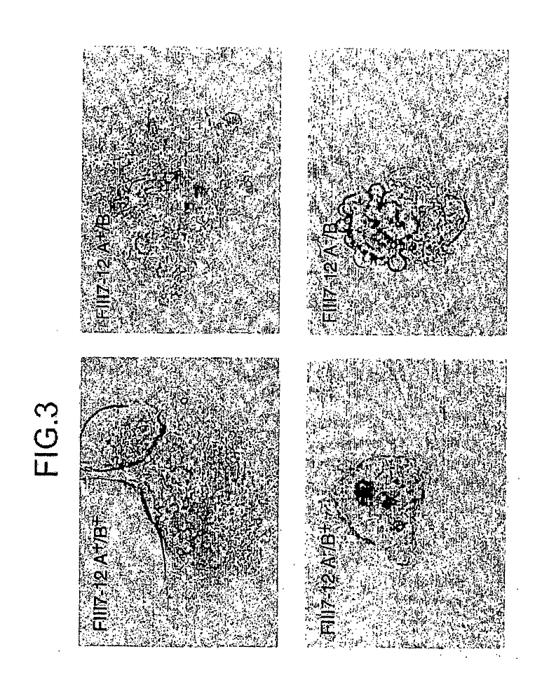

FIG. 3 are Photomicrographs of fully hatched human blastocysts cultured in 400 ml of complex serum-free culture medium on recombinant FN variants FIII7-12A+B+, FIII7-12A+B−, FIII7-12A−B+ and FIII7-12A−B−. Blastocysts exhibit varying degrees of trophoblast outgrowth.

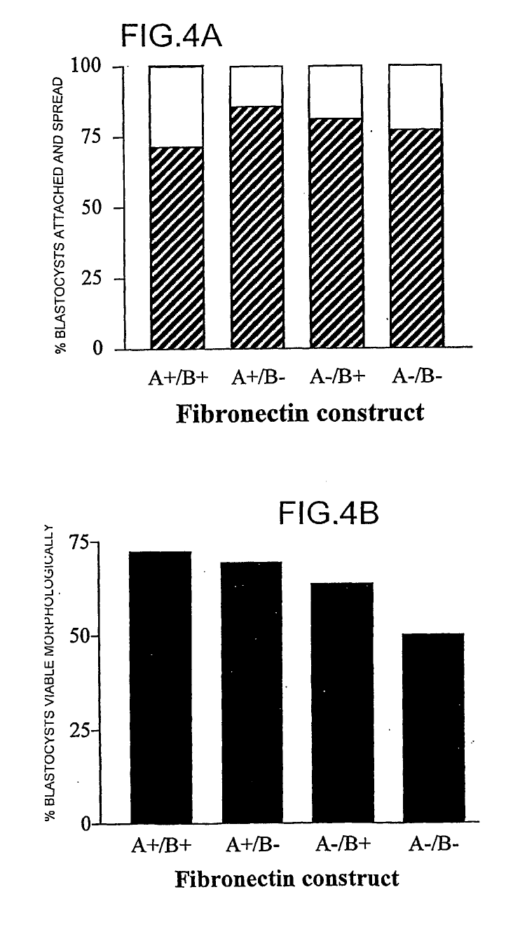

FIG. 4 shows behaviour of human blastocysts cultured on recombinant FN variants. A Percentage of fully hatched blastocysts adhering (white bars) and spreading (shaded bars) on fibronectin construct A+B+ (n=18), A+B− (n=13), A−B+(n=11) and A−B− (n=14). B Percentage of fully hatched blastocysts appearing morphologically viable after ˜24 h culture on fibronectin construct A+B+ (n=18), A+B− (n=13), A−B+(n=11) and A−B− (n=14). C Cumulative hCG production (mU/24 h) by fully hatched blastocysts adhering to fibronectin construct A+B+(n=10), A+B− (n=10), A−B+(n=6) and A−B− (n=8). Means are significantly different (P<0.05; one-way analysis of variance)

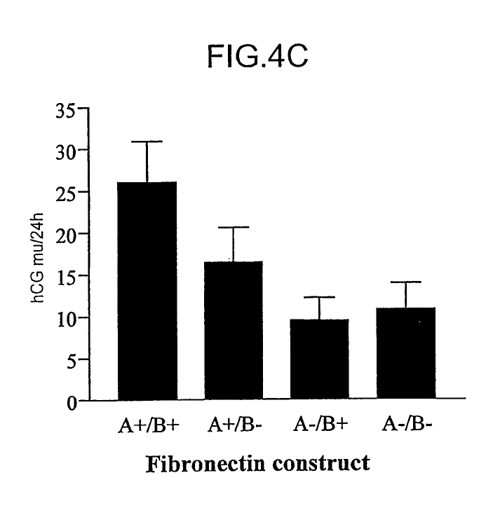

FIG. 5 shows blastocysts cultured on EDIIIA+/B+ FN stained for integrin subunits α5 and β1 and integrin heterodimers ανβ3 and ανβ5 showing trophoblast outgrowth. α5 and β1 localise to focal adhesion complexes whereas ανβ3 and ανβ5 remain more diffuse on the cell membrane.

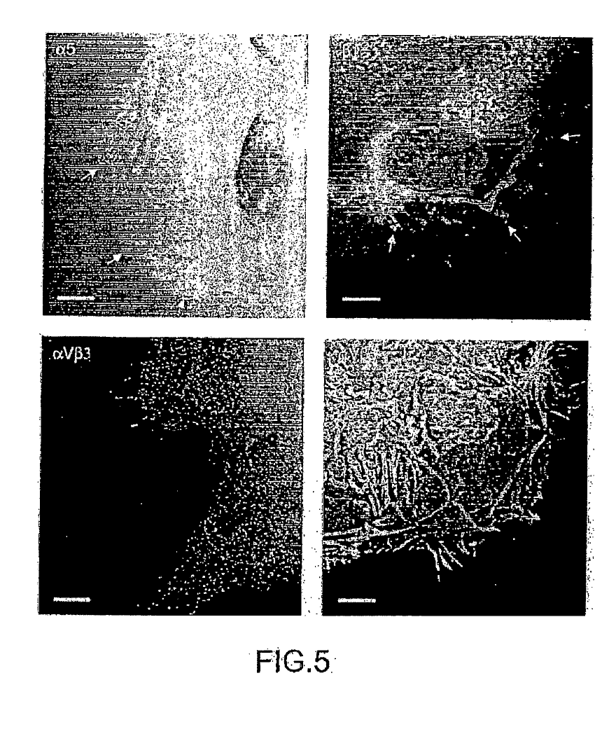

FIG. 6a) shows the sequence alignment of the mouse (upper) and human FIII9 module using the program ‘fasta’ (GCG package). Colons indicate identical amino acid pairs, with stops indicating conservative amino acid pairs and spaces indicating a non-conservative pair. Every tenth amino acid is noted with an upper dot.

FIG. 6b) is a ribbon diagram of the FIII9 modules. Atom co-ordinates were obtained from The Protein Data Bank (Berman et al., Nucleic Acids Res., 28, 235-242, 2000) (PDB ID: 1FNF) and imaged without modification using the program ‘Rasmol’ (www.umass.edu/microbio/rasmol). The b-strands are shown as cartoons (grey) and the three substituted residues are shown as ball and stick diagrams (black). The synergy loop faces away from the viewer (arrow).

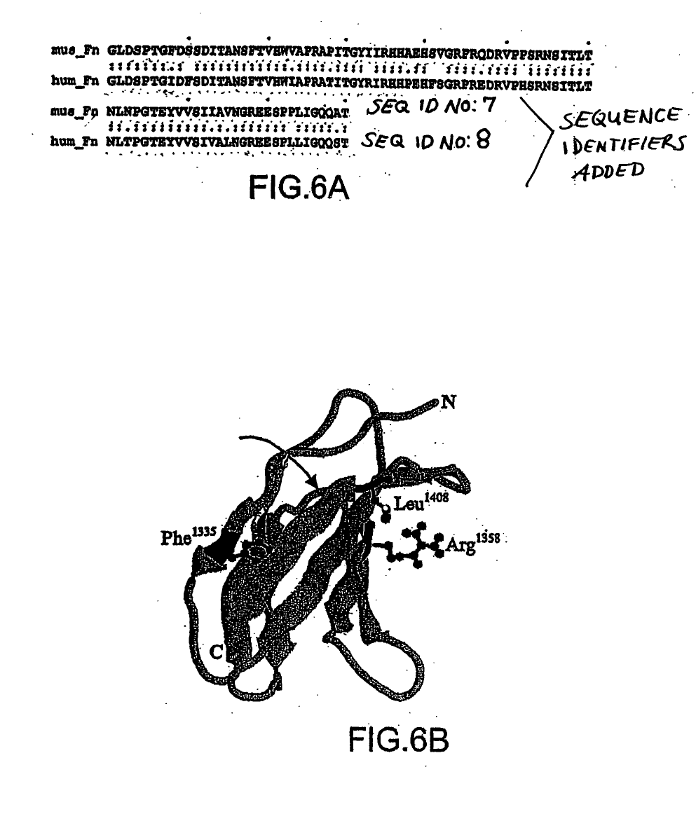

FIG. 7 shows the thermodynamic comparison of recombinant proteins. Equilibrium denaturation, shown as the % unfolded protein versus [GdnHCl], for the wild-type and variant FIII9-10 pairs (squares, wild-type; down triangles, Ile¹³⁵⁸ variant; up triangles, Pro¹⁴⁰⁸ variant; diamonds, Ile¹³⁵⁸-Pro¹⁴⁰⁸ variant). The two transition regions relate to the initial unfolding of the FIII9 module, followed by FIII10 unfolding. INSERT: ΔG as a function of [GdnHCl] for the first denaturation step in wild type and variant FIII9-10 recombinant proteins. Linear regression analysis was used to calculate the slope (m) and y-axis intercept (ΔG_{H2O}), presented in Table 1.

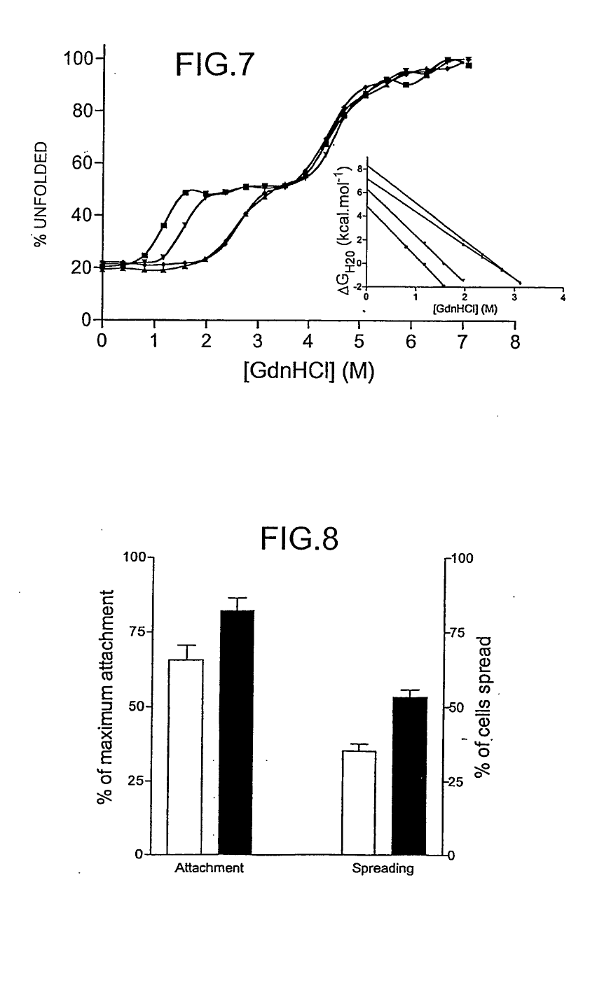

FIG. 8 shows cell attachment and spreading in response to cover slips coated with 0.38 μg/ml wild type FIII9-10 (white bars) or leucine to proline mutant FIII9-10 Pro¹⁴⁰⁸ (shaded bars).

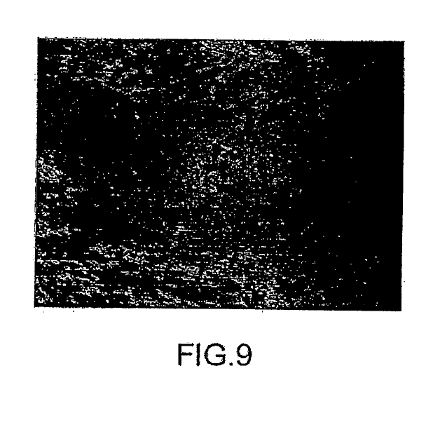

FIG. 9 shows a confluent layer of endometrial stromal cells with an implanting embryo in the centre, stained for EDIIIA+ fibronectin (red) (clone IST-9), and DAPI (blue) showing nuclei.

### EXAMPLE 1

### Role of EDIIIA and EDIIIB Splice Variants in the Embryo Implantation in the Endometrium

Endometrial Tissue Samples

Endometrial tissue samples were collected in accordance with the requirements of the Central Oxford Research Ethics Committee. Endometrial samples were obtained from patients aged 20-45 years undergoing hysterectomy for benign indications, who had a regular 26-33 day menstrual cycle and who had received no hormonal medication in the preceding three months. The stage of the menstrual cycle with respect to the last menstrual period was confirmed by histological examination of the endometrium using the criteria of Noyes et al (Noyes et al, Fertility Sterility, 1:3-25, 1950). Twenty-four samples of endometrium (4 early-, 5 late-proliferative; 6 early-, 5 mid-, 4 late-secretory) were obtained. Tissues were snap frozen in liquid nitrogen and stored at −80° C. until required for immunohistochemical analyses.

Embryo Collection and Culture

Ethical approval for this study was obtained from the Central Oxford Research Ethics Committee and the Human Fertilisation and Embryology Authority. All of the embryos utilised in this project were donated for research with informed consent from patients attending the Oxford Fertility Unit, John Radcliffe Hospital, for infertility treatment.

Embryo culture:—Ovarian stimulation, oocyte retrieval and insemination have been described previously (Dorkas et al., Hum. Reprod., 8, 2119-2127, 1993; Martin et al., Hum. Reprod., 13, 1645-1652, 1998). Embryos surplus to treatment and freezing were transferred on day 2 to 100 ml of a complex-serum free medium (CSFM3) supplemented with 1 mM HB-EGF and 2.5% HSA (Martin et al., Hum. Reprod., 13, 1645-1652, 1998), and overlaid with 1 ml of light paraffin oil (Sigma, UK). Embryos that had developed to the hatched blastocyst stage (day 6-7) were then transferred onto one of the recombinant fibronectin variants.

Cloning of Fibronectin Variant cDNAs and Protein Expression

Construction of EDIIIA/B variant constructs:—Four fibronectin variant cDNAs (see FIG. 1) encoding the region spanning the FIII7 to FIII12 domains were cloned into the bacterial expression vector pGEX2T (Pharmacia UK). The cDNAs encoding FIII9-12 (G¹³⁵⁷-E¹⁸¹²) (Kornblihtt et al., Nucleic Acids Res., 12, 5853-5969, 1984) were amplified in polymerase chain reactions (PCR) using pfu DNA polymerase with phosphorylated oligonucleotide primers GGGTCTTGATTCCCCAACTGG (forward) and TTATTACTCCAGAGTGGTGACAACA (reverse). Two templates, one EDIIIA+ cDNA (pFH111, Kornblihtt et al., Nucleic Acids Res., 12, 5853-5969, 1984) and one EDIIIA− (pFHL1, Kornblihtt et al., Nucleic Acids Res., 12, 5853-5969, 1984) were used. The amplified products were cloned into the Sma I site in pGEX2T, generating clones pGEXFIII9-12A+/−. A cDNA spanning FIII7-10. (p¹¹⁷³-T¹⁵⁴⁰) containing the EDIIIB domain was obtained by reverse transcriptase-PCR (RT-PCR) of placental RNA (prepared by a standard procedure, such as described in Sambrook et al, Molecular Cloning: A Laboratory Manual, 1989) using random primers and AMV reverse transcriptase. Amplification was achieved with the use of the primers GCCATTGTCTCCACCAACAAA (forward) and TTATTATGTTCGGTAATTAATGGAAA (reverse). The corresponding FIII7-10 EDIIB− cDNA was amplified from pFHL1 using the same primers. Amplified products were gel-purified using QIAEX II (Qiagen Ltd., Dorking, UK) according to the manufacturer's instructions and cloned into the Sma I site in pGEX2T. The sequences of the inserts were confirmed by dye terminator sequencing (performed by the DNA Sequencing Facility, Department of Biochemistry, University of Oxford). These clones were designated pGEXFIII7-10B+/−. The FIII7-12 variants were obtained by cleaving the pGEX2TFIII9-12A+ and pGEX2TFIII9-12A− with Eco RI, the site for which occurs once in the vector polylinker and once in FIII9, and inserting the gel-purified (as above) fragments into the Eco RI site in pGEXFIII7-10B+ and in pGEXFIII7-10B−. The sequences of the inserts in transformants were confirmed as above and the resulting four variant constructs designated pGEXFIII7-12A+B+, pGEXFIII7-12A+B−, pGEXFIII7-12A−B+ and pGEXFIII7-12A−B−.

Protein expression-GST fusion proteins FIII7-12A+B+, FIII7-12A+B−, FIII7-12A−B+ and FIII7-12A−B− (as represented in FIG. 1) were expressed and purified as described previously ref. Fusion proteins were dialyzed extensively against phosphate buffered saline (PBS) for use in the embryo attachment assays described below. Purity of the proteins was assessed by SDS-gel electrophoresis and integrity of the purified protein was confirmed by mass spectroscopy. The concentration of the protein was measured using the Bradford Assay kit (BioRad) according to the manufacturer's instructions. Variant proteins were tested for adhesive activity in cell attachment assays as described elsewhere ref

Functional Assays

Embryo culture on recombinant fibronectin fragments:—Hatched blastocysts were transferred to a single well of a 4-well plate containing a glass coverslip pre-coated overnight at 4° C. overnight with 50 mg one of the following recombinant fibronectin constructs: FIII7-12A+B+, FIII7-12A−B−, FIII7-12A−B+ and FIII7-12A−B−. Embryos were cultured for a mean of 48 h in 400 ml of CSFM3 supplemented with 2.5% HSA. After this time, embryos remaining attached to fibronectin were washed twice with prewarmed phosphate buffered saline (PBS) before fixation in 3% paraformaldehyde for 10-30 min. Following two further washes in PBS, embryos were stored in 1 ml of PBS at 4° C. for up to 48 h. The culture medium was collected and stored at −20° C. prior to analysis for hCG levels.

hCG assay:—The secretion of hCG by blastocysts into the culture medium was determined using a solid-phase, two-site fluoroimmunometric assay (Delfia hCG, Wallac, Milton Keynes, UK). The total amount of hCG produced by each embryo was expressed in mU.

Immunohistochemistry

An indirect immunofluorescence method was used to detect specifically EDIIIA⁺−, and the presence of EDIIIB⁺− FN, and total FN, using mAbs Ist-9, B-C1 and IST-4 respectively, in sections of endometrium and to detect integrin subunits in embryos cultured on fibronectin variants.

Endometrial tissue sections:—7 μm frozen sections were fixed in acetone for 10 min, blocked with 20% normal sheep serum for 20 min and then incubated with the primary antibodies in 10% normal human serum at room temperature for 1 h. A negative control was included by substituting the primary antibody with mouse IgG at 10 g/ml. Sections were washed for 5 min with 0.05% BSA in PBS and incubated for a further 40-45 min in the dark with fluorescein isothiocyanate-conjugated anti-mouse IgG diluted 1:65 with 0.05% BSA in PBS. Sections were washed in PBS and mounted using Vectashield mounting medium (Vector Laboratories).

Embryos:—Coverslip cultures of embryos on FN proteins were fixed at room temperature for 10 min with 3% paraformaldehyde in PBS followed by permeabilization for 10 min. The coverslips were then washed in 3% BSA in PBS for 5 min and blocked for a further 15 min using fresh 3% BSA solution. Incubations with primary and secondary antibodies were performed as described above. The coverslips were washed 3 times in PBS, inverted over Vectashield mounting medium containing DAPI on glass microscope slides and sealed. The following primary mAbs to integrin subunits, all diluted to 10 μg/ml in 1% BSA in PBS, were used: mouse anti CD49e (α5) (Serotec, clone SAM-1), anti CD51 (αV) (Coulter-Immunotech, clone AMF7) and anti CD61 (β3) (Becton Dickinson, clone RUU-PL 7F12). A negative control was included by substituting the primary antibody with mouse IgG at 10 g/ml. The secondary antibody used was either a fluorescein isothiocyanate-conjugated anti-mouse IgG (Sigma) diluted 1:65, in conjunction with 17 μM Texas Red-phalloidis (Molecular Probes) for visualisation of actin, or a Texas red-conjugated anti-mouse IgG (Jackson Immunoresearch Laboratories) diluted 1:75 (17 μg/ml) in 1% BSA in PBS.

Statistical Analyses

Fishers Exact test was used to compare morphological differences between the percentage of embryos cultured on the four recombinant fibronectin constructs. The area of embryo outgrowth and hCG secretion were analysed using the Unpaired Students t-test, and one way analysis of variance.

Results

Levels of EDIIIA+ and EDIIIB+ FN variants are elevated in the human endometrium at the time of implantation

The expression of EDIIIA+ and EDIIIB+ fibronectins in the human endometrium was analysed by immunohistochemistry (FIG. 2) using mAbs that recognise all forms of FN (IST-4), or that detect specifically EDIIIA+ and EDIIIB+ FN (IST-9 and BC-1, respectively). Glandular and lumenal epithelium was identified in adjacent sections by detection with anti-cytokeratin antibodies (FIG. 2, D and H). Fibronectin detected by IST-4 was abundant in the endometrium throughout the cycle whereas the EDIIIA+ and EDIIIB+ forms exhibited strict cell-type specific, cycle stage-dependent expression. In proliferative stage endometrium (FIG. 2, A, B, C and D), detection of fibronectin with the use of IST-4 showed abundant expression in the stroma, and in the ECM around the endothelium of the blood vessels and glandular epithelium (FIG. 2A). In contrast, levels of EDIIA+ and EDIIIB+ FN were very low and expression of EDIIIA+ and EDIIIB+ FN was restricted to the ECM surrounding the blood vessels in the proliferative stage (FIGS. 2B and C).

Levels of FN detected by IST-4 in endometrium obtained at the mid-secretory stage of the cycle (FIG. 2, E, F, G and H) were very similar to those in proliferative stage endometrium (compare FIG. 2, A with E). However, levels of EDIIIA+ and EDIIIB+ FN were elevated in the stromal ECM and the matrix surrounding the vascular endothelium and glandular epithelium compared to proliferative stage endometrium (compare FIG. 2B with F, and C with G). At high magnifications, staining of EDIIIA+ and EDIIIB+ fibronectins was particularly prominent in the epithelial ECM directly underlying the luminal edge of the endometrium and in the ECM of the stroma at the mid-secretory stage of the cycle (FIGS. 2I and J).

These results show that EDIIIA+ FN, and to a lesser extent EDIIIB+ FN, are specifically elevated in human endometrium at the time of implantation, suggesting function for EDIIIA+/B+ FN in implantation.

Recombinant EDIIIA+ and EDIIIB+ fibronectin fragments support embryo attachment and trophoblast outgrowth in vitro.

Having established that levels of EDIIIA+ and EDIIIB+ FN are elevated in the human endometrium at the time of implantation, the function of endometrial FNs was explored by testing the capacity of EDIIIA+/B+ FNs to support directly embryo attachment and trophoblast outgrowth in vitro. The ability of recombinant fragments of human FN spanning domains FIII7-12 and containing either EDIIIA or EDIIIB, or both, or neither (FIII7-12A+/B−; FIII7-12A−/B+; FIII7-12 A−/B−; FIII7-12 A+/B+; see FIG. 1) was tested. Hatched blastocysts cultured on coverslips coated with the recombinant fragments were assessed according to morphological criteria (see materials and methods). Blastocysts attached to and spread on all forms of FN to different degrees, (FIG. 3). Trophoblast outgrowth could be observed in the blastocysts. Some embryos showed signs of blebbing indicating apoptosis (eg FIG. 3, FIII7-12 A−/B−). Some of the embryos that attached to the recombinant FNs but did not undergo trophoblast outgrowth (FIG. 4A). X % did not attach to the FN coated coverslips. The morphology of the blastocysts differed according to the isoform (FIG. 4B). Approximately 75% blastocysts cultured on FIII7-12A+/B+ variant were morphologically viable, compared to 50% blastocysts cultured on FIII7-12A−/B− fibronectin. Viability on FIII7-12A+/B− and FIII7-12A−/B+was approximately 70% and 60%, respectively. Measurement of hCG in culture supernatants revealed that blastocysts cultured on EDIIIA+/B+ variant secreted highest amounts of hCG compared to blastocysts cultured on the other variants (FIG. 4C). Furthermore, blastocysts cultured on FIII7-12A+/B+ and FIII7-12A+/B− secreted significantly more hCG than those on isoforms without EDIIIA.

Trophoblast Spreading on EDIIIA+/B+ Fibronectins is Mediated by α5β1 Integrins

The integrin receptors that mediate blastocyst attachment and spreading on EDIIIA+/B+ FNs were determined by immunohistochemical localization of integrin subunits α5 and β1, and integrins ανβ3 and ανβ5, at the basal surface of the trophoblasts spreading on the different proteins. The staining patterns are shown in FIG. 5. Actin was visualized by the use of a phalloidin-fluorescence conjugate, and nuclei with DAPI. Trophoblast outgrowths on all fibronectin isoforms stained positive for α5, β1, ανβ3 and ανβ5 integrins. Staining for α5 was localised in structures resembling typical, large focal complexes, at the termini of actin filaments, at the edge of spreading trophoblasts (FIG. 5, α5), in addition to punctate staining across the cell membrane. Antibody to β1 revealed a similar pattern of staining to α5 (FIG. 5, β1). Staining for the integrin subunit αν and the heterodimer ανβ3 was markedly different from α5 and β1 (FIG. 5, ανβ3; ανβ5). Antibodies against both ανβ3 and ανβ5 resulted in a speckled staining pattern across the cell membrane, and there was no marked localisation in focal complexes at the edge of the cell as for α5 and β1. Staining for ανβ5 was similar to that of ανβ3 in that it was localised in a speckled pattern across the cell surface and was not localised to structures resembling focal adhesion complexes.

It has been demonstrated that, although a number of different integrins such as ανβ3, ανβ5 and α4 (data not shown) are expressed by trophoblast in blastocysts cultured on EDIIIA+/B+ FN, only α5β1 is localised in focal adhesion complexes. It is possible that the pattern of expression of other integrins represents a novel form of functional adhesion complex, but it is likely that trophoblast attachment outgrowth is mediated by α5β1.

hCG Secretion

Successful implantation of the blastocyst requires the embryo to undergo controlled proliferation, development, trophoblast invasion and differentiation: processes that rely upon ECM components. The secretion of hCG provides a reliable marker of embryo quality and integrity during these early stages of pregnancy. Present results data demonstrate that EDIIIA+ FN induces elevated levels of hCG during attachment and trophoblast outgrowth. Present results suggest that the expression of EDIIIA+ FN in the pregnant endometrium promotes the embryonic well being and cellular responses required for successful implantation.

### EXAMPLE 2

### Novel Variant FIII9-10 Peptides Derived from Human Fibronectin

Construction of pRSET-a Clones

The DNA sequence of hFIII9-10 was amplified from the plasmid pFH154 (Kornblihtt et al., Nucleic Acid Res., 12, 5853-5868, 1984) using the primers 5′-CGATGCGGTACCGCTAGCGGTCTTGATTCCCCAACTGG and 5′-CGCAAGCTTTTATGTTCGGTAATTAATGGAAATTG. The PCR product was digested with HindIII-KpnI (New England Biolabs) and ligated into HindIII-KpnI restricted pBluescript KS (Stratagene). Mutations were made following the Quickchange™ protocol (Stratagene). The +/−primers used to mutate Leu¹⁴⁰⁸ to Pro¹⁴⁰⁸ were GGCAGAGAGGAAAGTCCCCCATTGATTGGCCAAC and GTTGGCCAATCAATGGGGGACTTTCCTCTCTGCC, respectively. The +/− primers used to mutate Arg¹³⁵⁹ to Ile¹³⁵⁸ CCACCATCACTGGCTACATCATCCGCCATCATCC and GGATGATGGCGGATGATGTAGCCAGTGATGGTGG, respectively. The +/− primers used to mutate Phe¹³³⁵ to Ser¹³³⁵ CCAACTGGCATTGACTCTTCTGATATTACTGCC and GGCAGTAATATCAGAAGAGTCAATGCCAGTTGG, respectively.

Mutations were carried out sequentially until the three variants: hFIII9-10 Pro¹⁴⁰⁸, hFIII9-10 Ile¹³⁵⁸, hFIII9-10 Pro¹⁴⁰⁸ Ile¹³⁵⁸ were constructed. Each clone was digested with KpnI-NheI and the agarose-purified cassette ligated into the KpnI-NheI restricted expression vector pRSET-a (Invitrogen). The DNA sequence of all plasmid constructs was confirmed using Sanger DNA sequencing methodology (Biochemistry Dept., University of Oxford).

Expression and Purification of FIII Proteins in E. Coli

E. coli BL21(DE3)pLysS (Promega) were transformed with pRSET-a-hFIII9-10 (and variants thereof), grown to an OD₆₀₀ of 0.6 in LB containing ampicillin (100 mg ml⁻¹) and chloramphenicol (10 mg ml⁻¹) and induced with 0.1 mM isopropyl b-D-thiogalactopyranoside (Sigma Chemical Co.). Cells were harvested 3 h later, sonicated (3×20 s; Soniprep 150, Sanyo Gallenkamp), and clarified by centrifugation (20,000 g for 1 h) and filtration (1 mm Puradisc 25 AS, Whatman). The lysate was loaded onto a 10 ml column of Ni-NTA superflow (Qiagen) and recombinant protein was purified following the manufacturers instructions, with an added wash step using 70 mM imidazole.

Purity and Mᵣ of all proteins was assessed by SDS-PAGE, visualised with Coomassie blue. Protein concentration was calculated using absorption of the solution at 280 nm, with the extinction coefficient estimated using the program ‘peptidesort’ (Wisconsin package ver. 10.0, Genetics Computer Group, Madison, USA) using the procedure of Gill and von Hippel (1989).

Mass spectra were obtained for hFIII9-10 (obs 21486.02, calc 21485.80), hFIII9-10 Pro¹⁴⁰⁸ (obs 21470.36, calc 21469.73); hFIII9-10 Ile¹³⁵⁸ (obs 21443.73, calc 21442.74) and hFIII9-10 Pro¹⁴⁰⁸ Ile¹³⁵⁸ (obs 21427.31, calc 21426.70).

Equilibrium Chemical Denaturation

Equilibrium unfolding experiments were performed on recombinant FIII proteins incubated in 0 to ˜8 M Guanidine hydrochloride (GdnHCl) in 10 mM HEPES, 100 mM NaCl, pH 7.4. The molarity of the GdnHCl solution was calculated by the weight of solution (Pace et al., Protein Structure, a Practical Approach, page 299-321, 1997, IRL Press, Oxford). Protein samples were rapidly diluted 11 fold in GdnHCl and allowed to equilibrate for 10 min at 25° C. before measurement monitored by fluorescence at 350 (±1) nm, using an excitation wavelength of 278 nm on a Shimadzu RF5001PC spectrofluorimeter, held at 25° C. All GdnHCl denaturation curves were repeated independently and analysed using a linear extrapolation method (to fit the equation: ΔG_{obs}=ΔG_{H20}−m. [GdnHCl]) assuming a two-state unfolding mechanism as described by (Pace et al, 1997, as above).

Cell Attachment and Spreading Assays

Baby hamster kidney fibroblasts (BHK) cells were used in cell attachment and spreading assays and inhibition of cell spreading assays, carried out as described before (Mardon, H. J. & Grant, K., FEBS Lett., 340, 197-201, 1994).

Results

Overlap of primary sequences using ‘fasta’ (Wisconsin package ver. 10.0, Genetics Computer Group, Madison, USA) highlighted a total of 6 non-conservative amino acid substitutions and 10 conservative amino acid substitutions between the mouse-human FIII9 modules (FIG. 6). The overall sequence identity between the mouse and human FIII9 modules was 83.3%, comparing against a sequence identity of 86.2% between the FIII10 modules. Three amino acid substitutions, Ser¹³³⁵ for Phe¹³³⁵, Ile¹³⁵⁸ for Arg¹³⁵⁸ and Pro¹⁴⁰⁸ for Leu¹⁴⁰⁶, were made to the human FIII9 module on the basis of location and a desire to minimise alteration to the wild-type sequence. SDS-PAGE analysis of the soluble cell fraction showed that expression of the variant FIII9-10 constructs was equal to, or higher than, expression of hFIII9-10, except for constructs containing the Ser¹³³⁵ mutation where expression was not apparent. Evidence of expression of the Ser¹³³⁵ variants was found in the insoluble cell (pellet) fraction suggesting that substitution of Ser¹³³⁵ for Phe¹³³⁵ was detrimental to the conformational stability of FIII9. Therefore, Ser¹³³⁵ variants were not taken further in equilibrium denaturation and ell spreading experiments.

Pro¹⁴⁰⁸ Creates FIII9-10 Variants with Improved Stability

A two-state transition (N<<U) was assumed for the protein unfolding of FIII modules with the equilibrium constant defined as the ratio of fraction unfolded to fraction folded, according to the method of Pace and Scholtz (1997). It has been argued that this analysis underestimates errors as no error is assumed in the pre- and post-transition baselines (Santoro et al., Biochem., 27, 8063-8070, 1988). This was considered only to be a problem with respect to the post-transition baseline for FIII9 unfolding in variants hFIII9-10 Pro¹⁴⁰⁸ Ile¹³⁵⁸ and hFIII9-10 Pro¹⁴⁰⁸, which was a consequence of the increased stability of the FIII9 module in these variants. Evidence for a two-state transition has been provided previously by the observation that the kinetics of the recovery of the wild-type fluorescent and circular dichroism signals were effectively identical (Plaxco et al., Proc. Natl. Acad. Sci. USA., 93, 10703-10706, 1997). FIG. 7 shows two-step equilibrium denaturation curves for hFIII9-10 and variants hFIII9-10 Pro¹⁴⁰⁸, hFIII9-10 Ile¹³⁵⁸ and hFIII9-10 Pro¹⁴⁰⁸ Ile¹³⁵⁸. It has been demonstrated previously that the initial step represents the unfolding of FIII9 and the latter step represents the unfolding of FIII10; our values of ΔG_{H20}, m, and [GdnHCl]_{1/2} for hFIII9-10 (Table 1) are also in good agreement with that report (Spitzfaden et al., J. Mol. Biol., 265, 565-579, 1997). As expected, a large difference in thermodynamic stability was observed between the FIII9 and FIII10 modules, demonstrated by wild-type ΔG_{H20} values of 4.9 and 12.6 kcal mol⁻¹, respectively. None of the mutations effectively altered the stability of FIII10, the differences obtained in values of ΔG_{H20} and m can be accounted for in the error associated in the linear extrapolation of ΔG back to zero concentration GdnHCl.

Most noticeable was the increase observed between the free energy of unfolding (ΔG_{H20}) of the wild-type and Pro¹⁴⁰⁸ variant. Since ΔG_{H20} provides a measure of the conformational stability of the folded module, the Pro¹⁴⁰⁸ variant therefore appeared to stabilise the FIII9 conformation 1.5 fold. The Ile¹³⁵⁸ mutation also resulted in a modest increase in the conformational stability of FIII9 but when combined with Pro¹⁴⁰⁸ mutation in the double variant, a cumulative increase in the conformational stability was observed (ΔG_{H20} rising to 8.3 kcal mol⁻¹). The cumulative effect of the Pro¹⁴⁰⁸ and Ile¹³⁵⁸ mutations was not reflected in the GdnHCl unfolding curves and for this reason [GdnHCl]_{1/2} values for FIII9 in the Pro¹⁴⁰⁸ variant and double variant were both 2.6 M. Since the [GdnHCl]_{1/2} value for FIII9 in the Pro¹⁴⁰⁸ variant was two-three fold higher than for the wild-type, the denaturant clearly had less of an effect on the transition between the folded and unfolded states of this variant than would be predicted from its conformational stability. The dependence of the free energy change on denaturant concentration (m) for FIII9, which reflects the surface area exposed to solvent in the unfolded conformation of the module, dropped from 4.3 kcal mol⁻¹ M⁻¹ in the wild-type pair to 2.8 kcal mol⁻¹ M⁻¹ in the Pro¹⁴⁰⁸ variant. This suggests that substitution of Pro¹⁴⁰⁸ for Leu¹⁴⁰⁸ altered the mechanism of FIII9 unfolding and is in contrast to the substitution of Ile¹³⁵⁸ for Arg¹³⁵⁸ which resulted in only a modest loss of sensitivity to denaturant (m=4 kcal mol⁻¹ M⁻¹).

Substitution of Leu¹⁴⁰⁸ with Pro Enhances the Cell Adhesive Activity of FIII9-10

The adhesion of promoting activity of the mutant L1408P was determined in cell attachment and spreading assays. In comparison with wild type FIII9-10, both cell attachment and spreading were enhanced on surfaces coated with mutant L1408P at a range of concentrations (data not shown). At low coating concentrations (0.38 μg/ml) the increase in attachment and spreading activities was ˜25% and ˜50% respectively (FIG. 8), in accordance with the increased conformational stability observed for the L1408P mutant. Of the three amino acid mutations, Pro¹⁴⁰⁸ had a marked effect on both protein expression and conformational stability with the solution properties of hFIII9-10 Pro¹⁴⁰⁸ comparing very favourably to hFIII9-10. The hFIII9-10 Pro¹⁴⁰⁸ protein concentrated to align molarity without any visible aggregation-precipitation. Although the Pro¹⁴⁰⁸ variant was two-three fold more stable to GdnHCl than the wild-type ([GdnHCl]_{1/2}=2.6 M), its conformational stability was calculated to be only around 1.5 times as great (ΔG_{H2O} ˜7.2 kcal mol⁻¹). This discrepancy may be explained by taking into account m, which is seen to be less for both the Pro¹⁴⁰⁸ and Pro¹⁴⁰⁸-Ile¹³⁵⁸ variants, suggesting that introduction of Pro¹⁴⁰⁸ restricts the extent of unfolding seen in the wild-type FIII9 module. The location of this residue, which is predicted to lie at the boundary between the ‘F-G’ loop and ‘G’β-strand (Leahy et al., Cell, 84, 155-164, 1996), could also be of relevance. Sequence alignment of the FIII7, 8, 9 and 10 modules shows that a Pro residue is commonly found at the beginning or end of a predicted β-strand, possibly having some role in the folding of the common type III fibronectin module. Interestingly, three Pro-Pro pairs are seen in the mouse FIII9-10 pair, while none exist in the human FIII9-10 pair. Such a Pro-Pro pair would normally be expected to severely restrict the local flexibility of the protein backbone because of the narrow range of dihedral angles allowed by the Pro residue. However, refolding of the Pro-rich human FIII10 module proceeds very rapidly (Plaxco et al., J. Mol. Biol., 270, 763-770, 1996) and this module is also noted for its excellent solution and stability characteristics (Spitzfaden et al., J. Mol. Biol., 265, 565-579, 1997). These Pro-Pro pairs may therefore contribute to the superior stability of the mouse, over the human, FIII9-10 pair and also explain why hFIII9-10 Pro¹⁴⁰⁸ showed such a marked improvement in solubility and stability.

Cell attachment and spreading assays revealed that the Ile¹³⁵⁸ and Pro¹⁴⁰⁸ mutations enhanced biological function, when compared to wild-type hFIII9-10. The present results show that the conformational stability of the FIII9 module is increased using minimal amino acid substitutions to maintain activity and, moreover, the solubility of the human FIII9-10 pair is improved as the first step to its structural characterisation.

### EXAMPLE 3

### Screen for Compounds Modulating EDIIIA+ and/or EDIIIB+ Production

a) Western Blotting Assay

1. Plate cells on a 25 cm² tissue culture in DMEM, penecillin/streptomycin, 10% FCS.

2. Incubate cells at 37° C. overnight.

3. Protein is precipitated from culture supernatants by the addition of 2-3 volumes of ice-cold 95% ethanol.

4. The precipitated protein from individual replicate 80 cm² flasks is collected by centrifugation at 2000 revs/min for 5 min and resuspended in 100 μl protein sample buffer (Laemmli, 1970).

5. Cells are lysed with 0.02 M NH₄OH (Gospodarwicz et al., 1984) and the lysate collected.

6. The material remaining attached to the flask washed extesively with PBS, and resuspended in 400 μl protein lysis buffer (1% Triton X-100; 0.5% sodium deoxycholate; 0.5% SDS; 0.1% NaCl; 5 mM MgCl₂; 50 mM Tris-HCl, pH 7.6) containing 3 mM phenylmethylsulfonyl fluoride, and 1 g/ml (w/v) each of aprotinin, leupeptins and pepstatin.

7. Proteins are precipitated and collected as above and resuspended in 100 μl protein sample buffer.

8. Protein samples are electrophoresed through a 4% polyacrylamide stacking gel and 6% polyacrylamide resolving gel containing 0.1% SDS (Laemmli, 1970) and transferred onto nitrocellulose filters (Towbin et al., 1979).

9. Actin, the 60 kDa actin-related protein, and FN variants, are detected with the ECL Western Blotting system (Amersham), according to the manufacturer's instructions with the use of anti-actin mAbs (Sigma); mAbs IST-4; IST9 or BC-1; sheep anti-mouse IgG conjugated to horseradish peroxidase (HRP; sigma, UK). Prestained protein markers (Gibco BRL, UK) lysozyme (15 kDa); β-lactglobulin (18 kDa); carbonic anhydrase (28 kDa); ovalbumin (43 kDa); BSA (71 kDa); phosphorylase b (100 kDa) and myosin (H-chain (218 kDa). Filters are exposed to X-ray film and densitometric analysis of images performed.

b) ELISA Test using IST-9 or BC-1

Test compounds are tested in the a modification of the ELISA assay described above:—

1. Plate cells in a 96-well tissue culture plate in DMEM, penecillin/streptomycin, 10% FCS.

2. Incubate cells at 37° C. overnight.

3. Add cytokines/growth factors. (10 ng/ml) or other known or novel modulators and incubate for 24 hr at 37° C. in DMEM F12 (minus phenol red), p/s, 2% FCS.

4. Aspirate off supernatant and wash cells ×2 with PBS (with plate-washer).

5. Lyse and “empty” cells with 0.02 μM NH₄OH.

6. Wash cells 10× with 100 μl PBS

7. Peroxidase treatment: treat cells for 30 min. at RT with 0.6% H₂O₂ in 40% MeOH/PBS.

8. Wash 3-5 min in PBS ×2.

9. Block with 3% BSA in PBS ×2 (once for 5 minutes and once for 30 minutes).

10. Wash ×1 with PBS.

11. Primary antibody: add IST-9 or BC-1 at 100 ng/ml (1:2500 dilution). Leave for 1 hr at RT.

12. Wash ×3 in PBS.

13. Secondary antibody: add rabbit anti-mouse-HRP (Sigma) at (1:1000 dilution). Leave for 1 hr at RT.

14. Wash ×2 in PBS.

15. Develop by dissolving one silver and one gold tablet from SigmaFast o-phenyldiamine diHCl in 20 ml dH₂O; add 100 μl l per well.

16. Stop reaction after 20 min. with 100 l 0.5M H₂SO₄. Read plate at 490 nm.

17. Wash plate thoroughly with PBS.

18. Add 100 μl 0.1% crystal violet solution for 15-30 min. at RT.

19. Wash plate thoroughly with water.

20. Add 100 μl l MeOH to each well.

21. Read plate at 600 nm.

### EXAMPLE 4

### Protocol for Delivery of Agent as a Fusion Protein of FIII9′-10 to Integrin-Expressing Cells

The efficiency of a FIII9-10 variants, in particular FIII9′-10, as a delivery agent to integrin expressing cells is assessed as follows:

The fused agent to be delivered can be a protein or a chemical, including cell-specific toxin (e.g. anti-cancer) or growth factor; expressing integrin can be a cancerous cell.

1. Generate his-taged FIII9′-10 agent X fusion protein. Alternatively, it is also possible to couple FIII9′-10 variant with site-specific non-protein chemical.

2. Coat sterile 96 well plates (flat-bottomed, cell culture grade with doubling) dilutions of FIII9′-10-X and incubate 16-24 h at 4° C.

3. On the day of the assay, aspirate protein using multi-channel pipettor and gently wash each well three times with PBS.

4. Block non-specific binding to the plate by incubating with 1% (10 mg ml⁻¹) BSA in PBS for 1 h at 37° C. Wash plate as before.

5. Equilibrate plate with serum-free medium (50 μl in each well) by incubating at 37° C. in 5% CO₂ for 30 minutes including doubling dilutions of FIII9′-10 to the wells in a constant total volume of medium.

6. Meanwhile, wash cells in warm PBS, then trypsinize and quench with serum-containing medium. Collect cells by centrifugation then resuspend in 10 ml warm PBS and spin again.

7. Resuspend the cells in warm serum-free medium (2-10 ml), remove an aliquot for counting and incubate the remainder in a 5% CO₂ incubator at 37° C. for 10 minutes. Use a Universal container and leave the lid loose to allow gaseous exchange.

8. Dilute cells to 2×10⁵ ml⁻¹ with serum-free medium, and rest for a further 5 minutes in a 5% CO₂ incubator at 37° C.

9. Plate 10⁴ cells into each well. Disperse clumps and ensure even distribution by gently pipetting up and down in the wells. Incubate at 37° C. in a 5% CO₂ incubator for one hour (or longer, if depending on cell type and assay to be performed) and perform assay.

The efficiency of the delivery can be assessed by testing cell adhesion and/or cell proliferation, as follows:

i) Adhesion Assay

1. Gently wash cells with warm PBS (use 8-channel pipettor to aspirate) and fix with 100 μl 4% glutaraldehyde/4% formaldehyde in PBS.

2. Count spread vs round. When finished, remove fixative and stain cells with 0.1% crystal violet for ˜10 minutes. Wash off really well in tap water. Solubilize dye with 200 μl methanol and read at 570 nm in the microtitre plate reader.

ii) Proliferation Assay

1. Plate human endometrial stromal cells (10⁴ cells/well) in 96 well plate in DMEM supplemented with 10% FCS, 100 IU/ml penicillin and 100 ng/ml of streptomycin and incubate for 24 hours.

2. Change the medium to serum-free and incubate for 18 hours.

3. Add 1 μCi H³-thymidine (Amersham Pharmacia Biotech, UK) to each well for the last 4 hour's of incubations and wash cells 3 times in PBS, harvest, and determine the amount of incorporated H³-thymidine using a βplate counter (Wallac Ltd., Finland).

### EXAMPLE 5

### Protocol for Testing Inhibitory Effect of FIII9-10 Variants, in Particular FIII9′-10, on Adhesion of Cell Types or Human Embryos to Fibronectin

1. Coat sterile 96 well plates (flat-bottomed, cell culture grade) with 10 μg/ml fibronectin and incubate 16-24 h at 4° C.

2. Passage cells to be sub-confluent at the time of the assay.

3. On the day of the assay, aspirate protein using multi-channel pipettor and gently wash each well three times with PBS.

4. Block non-specific binding to the plate by incubating with 1% (10 mg.ml⁻¹) BSA in PBS for 1 h at 37° C. Wash plate as before.

5. Equilibrate plate with serum-free medium (50 μl in each well) by incubating at 37° C. in 5% CO₂ for 30 minutes including doubling dilutions of FIII9′-10 to the wells in a constant total volume of medium.

6. Meanwhile, wash cells in warm PBS, then trypsinize. Allow the cells to sit in trypsin for the bare minimum amount of time necessary for detachment before quenching with serum-containing medium. Collect cells by centrifugation then resuspend in 10 ml warm PBS and spin again.

7. Resuspend the cells in warm serum-free medium (2-10 ml), remove an aliquot for counting and incubate the remainder in a 5% CO₂ incubator at 37° C. for 10 minutes. Use a Universal container and leave the lid loose to allow gaseous exchange.

8. Dilute cells to 2×10⁵ ml⁻¹ with serum-free medium, and rest for a further 5 minutes in a 5% CO₂ incubator at 37° C.

9. Plate 10⁴ cells (50 μl) into each well. Disperse clumps and ensure even distribution by gently pipetting up and down in the wells. Incubate at 37° C. in a 5% CO₂ incubator for one hour (or longer, depending on cell type).

10. Gently wash cells with warm PBS (use 8-channel pipettor to aspirate) and fix with 100 μl 4% glutaraldehyde/4% formaldehyde in PBS.

11. Count spread vs round. When finished, remove fixative and stain cells with 0.1% crystal violet for ˜10 minutes. Wash off really well in tap water. Solubilize dye with 200 μl methanol and read at 570 nm in the microtitre plate reader.

### EXAMPLE 6

### Diagnostic Test

The following diagnostic test is aimed to confirm whether EDIIIA+ is deficient during the implantation window in the endometrium of women with infertility or sub-fertility. The implantation is defined as days 6-8 post the day of the LH surge.

Generally, an endometrial sample suitable for carrying out the present test fulfills the following requirements:

Patient should preferably have ovulation confirmed; the sample should preferably correspond to the “implantation window” time; generally the “implantation window” time can be based on a regular menstrual pattern as approximately seven days before the expected first day of the menstrual period; or, alternatively, it can be based on using the mid-cycle leutenising hormone (LH) urine test (Clear-Plan Styx) to identify LH surge, starting at day 12 (until the surge is identified).

Generally, the sample can be taken by pipelle biopsy.

1. The endometrial sample is fixed in formaldehyde and processed for paraffin wax embedding. Sections are cut and EDIIIA+ fibronectin detected with the use of primary antibodies (IST-9) optimally diluted in PBS and applied to the sections for one hour at room temperature. Control staining is performed with the same antibodies pre-incubated with the corresponding control peptides.

2. Sections are washed in three changes of PBS and incubated in peroxidase conjugated anti-goat or anti-mouse IgGs for one hour at room temperature.

3. Sections are washed as above and bound antibodies are detected with HRP substrate anti-trans-membrane monoclonal antibodies and peroxidase detection system.

4. Correlate the appearance of EDIIIA/B+ fibronectin with endometrial receptivity and pregnancy success.

### EXAMPLE 7

### Mutant Fibronectin Integrin-Binding Fragment FIII9-10 L1408P Inhibits Embryo Implantation in a Physiological Relevant Screen

Protocol

Endometrial tissues at different stages of the menstrual cycle were obtained with approval from the Oxfordshire Research Ethics Committee (OXREC) from women aged 20-49 years undergoing hysterectomy for benign indications or sterilization. Patients had a regular 26-33 day menstrual cycle, and had received no hormonal medication in the preceding three months.

Endometrial stromal cells were isolated with the use of a method described previously (Fernandez-Shaw et al., 1992). Briefly, endometrial tissue was cut into small pieces and digested in 330 U/ml cbllagenase type I (Worthington Biochemical Corporation, New Jersey, USA) in Dulbecco's modified Eagle's medium (DMEM) for one hour at 37° C. Stromal cells were separated from intact glands by filtration of the digested tissue through a 40 mm gauze (Lockertex, Warrington, UK). The stromal cells in the filtrate were purified by centrifugation through a 25-60% percoll step gradient, diluted in PBS, pelleted by centrifugation and resuspended in PBS. The cells were plated into 75 cm² tissue culture flasks (10⁶/flask) maintained in DMEM supplemented with 10% heat-inactivated foetal bovine serum and 50 IU/ml-50 μg/ml penicillin-streptomycin at 37 C in a humidified environment with 5% carbon dioxide in air. Stromal cells were used between passages 2 and 10. The stromal cell cultures consistently yielded at least 92% purity as assessed by expression of the stromal cell marker Thy-1. The cells were plated onto 13 mm diameter glass coverslips (Chance Propper Ltd.) size 0 for the implantation model experiments.

Embryo Collection and Culture

Ethical approval for this study was obtained from 0xREC and a research license was obtained from the Human Fertilisation and Embryology Authority. Embryos were donated for research with informed consent from patients attending the Oxford Fertility Unit, John Radcliffe Hospital, for in vitro fertilisation treatment.

Embryo culture:—Ovarian stimulation, oocyte retrieval, insemination, and grading of the quality of day 2 embryos were performed as described previously (Dokras et al., 1993). Grade A or B embryos donated for research were transferred to 100 ml of a complex-serum free medium (CSFM3) supplemented with 1 mM HB-EGF and 2.5% HSA (Martin et al., 1998), and overlaid with 1 ml of light paraffin oil (Sigma, UK). Embryos were maintained in culture and those that developed to the hatched blastocyst stage (day 6-7) were then transferred onto endometrial stromal cell cultures.

Embryo-Endometrial Stromal Cell Co-Culture

Hatched blastocysts were cultured on a confluent layer of stromal cells on a 13 mm coverslip in a single well of a 4-well plate. The embryo-stromal cell co-cultures were maintained in 500 ml pre-equilibrated DMEM containing 5% human serum albumin and 25 mM either FIII9-10 L14508P or FIIIB-9 (Altroff et al, 2001) up to day 9 post-insemination. The cultures were subjected to time-lapse video microscopy with the use of a Leica DMIRB inverted microscope (Leica, UK) and Photonics Coolview camera (Improvision, UK) in a microscope incubator (Solent Scientific, UK) maintained at 37° C. and 5% CO₂. Images were captured at 24 and 48 hours and processed using Openlab software (both from Improvision, UK). Embryos were assessed for attachment to, and invasion through the stromal layer. At the end of the culture period the culture medium was collected and stored at −20° C.

Results

FIII9-10L1408P Inhibits Invasion of Embryos into the Endometrial Stromal Layer

Three of four embryos cultured in the presence of FIII8-9 invaded the stromal layer. Two of the eight embryos cultured in the presence of FIII9-10L1408P invaded the stromal cell layer. The remaining six embryos attached to the upper surface of the stromal cell layer but did not penetrate the layer.

### Reference

Altroff H, van der Walle C F, Asselin J, Fairless R, Campbell I D Mardon H J (2001) The eighth FIII domain of human fibronectin promotes integrin a5b1 binding via stabilization of the ninth FIII domain J Biol Chem, 276, 420, 38885-38892
   Sequence Listing
SEQ ID NO: 1 nucleotide sequence which encodes the FIII9-10 Pro¹⁴⁰⁸ protein.
SEQ ID NO: 2 amino acid sequence of FIII9-10 Pro¹⁴⁰⁸.
SEQ ID NO: 3 nucleotide sequence which encodes the FIII9-10 Ile¹³⁵⁸ protein.
SEQ ID NO: 4 amino acid sequence of FIII9-10 Ile¹³⁵⁸.
SEQ ID NO: 5 nucleotide sequence which encodes he FIII9-10 Pro¹⁴⁰⁸ Ile¹³⁵⁸ protein.
SEQ ID NO: 6 amino acid sequence of FIII9-10 Pro¹⁴⁰⁸ Ile¹³⁵⁸.
   Accession Numbers:
Human fibronectin:
cDNA-GenBank: X02761
protein-swissprot: P02751; D

1. A method for assessing fertility status in a female patient, said method comprising:
determining the level of fibronectin isoform EDIIIA+ and/or ED111B+ in a isolated endometrial sample, within implantation window time for the patient;
correlating the appearance of said fibronectin isoform with fertility status.

2. The method of claim 1, wherein the fibronectin isoform is EDIIIA+.

3. The method according to claim 1, wherein said method is based upon an ELISA assay.

4. A method for identifying compounds which are capable of modulating the production of fibronectin isoform EDIIIA+ and/or ED111B+ which method comprises:
contacting an EDIIIA+ and/or ED111B+ producing cell with said test compound;
determining the effect of the test compound on the amount of said fibronectin isoform and thereby identifying a compound which modulates the production of EDIIIA+ and/or ED111B+.

5. The method of claim 4, wherein said method is based upon Western-Blotting or ELISA assays.

6. The method according to claim 4, wherein the fibronectin isoform is EDIIIA+ fibronectin.

7. The method according to claim 4, wherein the cell is an isolated human female endometrial stromal cell.

8. A medicament comprising a protein or peptide comprising the amino sequence of SEQ ID NO:2, SEQ ID NO:4 or SEQ ID NO:6 or a biologically active portion thereof.

9. Use of a protein or peptide comprising the amino acid sequence of SEQ ID NO:2, SEQ ID NO:4 or SEQ ID NO:6 or a biologically active portion thereof in the manufacture of a medicament for inhibiting adhesion of cell types to fibronectin.

10. Use of a protein or peptide as claimed in claim 9 in the manufacture of a medicament for use as an anti-angiogenic agent and/or anti-tumorigenic agent.

11. Use of a protein or peptide as claimed in claim 9 comprising the amino acid sequence shown in SED ID NO.2.

12. A method of inducing contraception in a mammalian female comprising administering to said female a protein or peptide comprising the amino acid sequence of SEQ ID NO.2, SEQ ID NO.4 or SEQ ID NO.6 or a biologically active portion thereof.

13. The method of claim 12 wherein said protein comprises the amino acid sequence shown in SEQ ID NO.2.

14. A method as claimed in claim 12 wherein said mammalian female is a human female.

15. Use of a protein or peptide comprising the amino acid sequence of SEQ ID NO:2, SEQ ID NO:4 or SEQ ID NO:6 or a biologically active portion thereof as a delivery agent to deliver an agent selected from a biologically active ingredients, a cytotoxic agent an imaging agent.

16. Use according to claim 15, wherein said protein or peptide delivers said agents to integrin-expressing cells.

17. Use according to claim 15, wherein the biologically active ingredients are selected from the group comprising agents capable of interacting with integrins.

18. A pharmaceutical composition comprising a protein comprising the amino acid sequence of SEQ ID NO:2, SEQ ID NO:4 or SEQ ID NO:6 or a biologically active portion thereof attached to a pharmacologically active agent, a cytotoxic agent or an imaging agent and a pharmaceutically acceptable carrier or diluent.

19. A protein comprising the sequence of amino acids chosen from the group consisting of SEQ ID NO:2, SEQ ID NO:4 or SEQ ID NO:6 or a sequence of amino acids which differs from that set forth in SEQ ID NO: 2, SEQ ID NO:4 or SEQ ID NO:6 only in conservative amino acid changes.

20. A protein or peptide comprising a sequence of amino acids as shown in SED ID NO.2 or a sequence of amino acids which differs from that shown in SEQ ID NO.2 only in conservative amino acid changes.

21. A nucleic acid comprising a sequence of nucleotides which encodes the protein claimed in claim 19.

22. A nucleic acid comprising the sequence of nucleotides chosen from the group consisting of: SEQ ID NO: 1, SEQ ID NO:3 or SEQ ID NO:5 or a fragment thereof.

23. An expression vector comprising the nucleic acid of claim 21.

24. A host cell or organism transformed or transfected with the expression vector of claim 23.

25. An antibody which is capable of specifically binding to the protein claimed in claim 19 or an epitope thereof.

26. A nucleic acid probe which is capable of hybridizing to the nucleic acid of claim 21 under conditions of high stringency.

27. An antisense nucleic acid which is capable of hybridizing to the sequence of nucleotides chosen from the group consisting of SEQ ID NO:1, SEQ ID NO:3 or SEQ ID NO:5 under conditions of high stringency.

28. A antisense nucleic acid according to claim 27, wherein said sequence of nucleotides is SED ID NO:1.

29. A method for producing a pharmaceutical composition suitable for treating infertility in a female, or suitable for use as a contraceptive agent in a female, which method comprises:
a) carrying out a compound screening method as claimed in claim 4; and
b) formulating any compound identified as capable for modulation of production of fibronectin isoform EDIIIA+ and/or EDIIIB+ into a pharmaceutical composition with a pharmaceutical acceptable carrier or diluent.

30. A method of inducing contraception in a mammalian female which comprises administering to said female and inhibitor of production of fibronectin isoform EDIIIA+ and/or ED111B+.

31. A method for enhancing fertility in a mammalian female which comprises administering to said female an enhancer of production of fibronectin isoform EDIIIA+ and/or ED111B+.

32. A method as claimed in claim 30 wherein said mammalian female is a human female.

33. Use of an inhibitor of production of fibronectin isoform EDIIIA+ and/or ED111B+ in the manufacture of a composition for inhibiting angiogensis and/or tumourgenesis.

34. Use of an enhancer of production of fibronectin isoform EDIIIA+ and/or ED111B+ in the manufacture of a composition for the treatment of human female infertility.

35. A pharmaceutical composition which comprises an enhancer of production of fibronectin isoform EDIIIA+ and/or ED111B+ and a pharmaceutically acceptable carrier or diluent.

36. A contraceptive composition which comprises an inhibitor of fibronectin isoform EDIIIA+ and/or ED111B+ productions and a pharmaceutically acceptable carrier or diluent.
